(19) United States
(12) Patent Application Publication
(10) Pub. No.: US 2024/0318154 A1
(43) Pub. Date: Sep. 26, 2024

(54) GENE THERAPY FOR THE TREATMENT OF SEVERE COMBINED IMMUNODEFICIENCY (SCID) RELATED TO RAG1

(71) Applicants: CELLECTIS, Paris (FR); Albert-Ludwigs-Universitat Freiburg, Freiburg (DE)

(72) Inventors: Toni CATHOMEN, Freiburg (DE); Tatjana CORNU, Freiburg (DE); Julia KLERMUND, Freiburg (DE); Manuel RHIEL, Freiburg (DE); Julia ROSITZKA, Freiburg (DE); Philippe DUCHATEAU, Paris (FR); Alexandre JUILLERAT, Paris (FR)

(21) Appl. No.: 18/561,938

(22) PCT Filed: May 20, 2022
(86) PCT No.: PCT/EP2022/063764

§ 371 Date: Nov. 17, 2023

(30) Foreign Application Priority Data
May 20, 2021 (DK) PA202170258

(51) Int. Cl. C12N 9/22; A61K 35/28; C07K 14/47; C12N 5/0789; C12N 15/90

(52) U.S. Cl. CPC C12N 9/22; A61K 35/28; C07K 14/4705; C12N 5/0647; C12N 15/907; C12N 2510/00

(57) ABSTRACT

The present invention generally relates to the field of genome engineering (gene editing), and more specifically to gene therapy for the treatment of Severe Combined Immunodeficiency (SCID) related to RAG1. Particularly, the present invention pertains to the treatment of RAG1 deficiency in long-term repopulating hematopoietic stem cells (HSCs). The present invention provides means and methods for genetically modifying HSCs involving gene editing reagents, such as TALE-nucleases, that specifically target a non-functional endogenous RAG1 gene, comprising at least one mutation causing Severe Combined Immunodeficiency (SCID), thereby allowing the restoration of the normal cellular phenotype. The present invention also provides engineered RAG1-edited HSCs comprising an exogenous sequence comprising a nucleic acid sequence encoding a functional RAG1 protein which is integrated in said HSCs' genome into a non-functional RAG1 endogenous locus, resulting in the expression of a functional RAG1 polypeptide. The present invention further provides populations of cells comprising said engineered HSCs, pharmaceutical compositions comprising said engineered HSCs or populations of cells, as well as their use in gene therapy for the treatment of Severe Combined Immunodeficiency (SCID) related to RAG1.

### CROSS REFERENCE TO RELATED APPLICATIONS

This application is a national stage application of PCT/EP2022/063764, filed May 20, 2022, which claims priority to Denmark Application No. PA202170258, filed May 20, 2021, each of which are hereby incorporated by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 201,605 Byte ASCII (Text) file named “42476-251-ST25.TXT,” created on Oct. 4, 2023.

### FIELD OF THE INVENTION

The present invention generally relates to the field of genome engineering (gene editing), and more specifically to gene therapy for the treatment of Severe Combined Immunodeficiency (SCID) related to RAG1. Particularly, the present invention pertains to the treatment of RAG1 deficiency in long-term repopulating hematopoietic stem cells (HSCs). The present invention provides means and methods for genetically modifying HSCs involving gene editing reagents, such as TALE-nucleases, that specifically target a non-functional endogenous RAG1 gene, comprising at least one mutation causing Severe Combined Immunodeficiency (SCID), thereby allowing the restoration of the normal cellular phenotype. The present invention also provides engineered RAG1-edited HSCs comprising an exogenous sequence comprising a nucleic acid sequence encoding a functional RAG1 protein, which is integrated in said HSCs' genome into a non-functional RAG1 endogenous locus, resulting in the expression of a functional RAG1 polypeptide. The present invention further provides populations of cells comprising said engineered HSCs, pharmaceutical compositions comprising said engineered HSCs or populations of cells, as well as their use in gene therapy for treatment of Severe Combined Immunodeficiency (SCID) related to RAG1.

### BACKGROUND OF THE INVENTION

The genetic basis of more than 18 different forms of severe combined immunodeficiency (SCID) has been identified. In all cases, the genetic defect leads to a failure of T cell development or T cell function [1]. The different defects can be categorized according to pathways affected or by the specific immunological phenotype arising from the mutation. The distribution of the different genetic defects amongst the SCID population show that X-linked SCID (X-SCID), which arises most frequently from mutations in the IL2RG gene, accounts for approximately 40-50% of SCID phenotypes. The second most frequent SCID form, accounting for about 30% of SCID cases, results from null mutations in the recombination activating genes RAG1 and/or RAG2. The prevalence is about 1 to 9 in 100.000 births. The patients present with neonatal onset of life-threatening, severe, recurrent infections by opportunistic fungal, viral and bacterial microorganisms, as well as skin rashes, chronic diarrhoea, failure to thrive and fever. On the cellular level, RAG1/2 deficiency results in less than 1% of wild type V(D)J recombination activity in B and T cells. Consequently, immunologic observations include profound T- and B-cell lymphopenia, normal NK counts and low or absent serum immunoglobulins. Hypomorphic RAG mutations, which impair but do not abolish V(D)J recombination activity, lead to Omenn's Syndrome, a severe immunodeficiency associated with erythroderma, eosinophilia, chronic colitis, lymphadenopathy and profound immunodeficiency [2].

Although it has been shown that lentiviral based gene transfer into murine hematopoietic stem cells (HSCs) can correct the disease in mouse models to some extent [3,4], the natural spatiotemporally tightly regulated expression of RAG1/2 could not be reproduced with these viral gene transfer approaches. The resulting incomplete thymic reconstitution and a disease phenotype similar to human Omenn's Syndrome revealed the dilemma in finding the balance between genotoxicity and avoiding RAG deficiency associated autoimmunity. Moreover, lentiviral vectors show a non-random integration pattern in the human genome and have the potential to dysregulate gene expression. It is therefore critical to develop and test nuclease technologies for effective editing of RAG1/2 in primary human HSCs for future clinical use. This is particularly important given that physiologically regulated expression is necessary for full correction of the RAG defect.

Accordingly, there is an ongoing need for novel therapy approaches to treat Severe Combined Immunodeficiency (SCID).

### SUMMARY OF THE INVENTION

The present invention addresses this need by providing an improved gene therapy approach to treat Severe Combined Immunodeficiency (SCID) related to RAG1, allowing the correction of RAG1 deficiency in HSCs, notably in long-term repopulating HSCs (LT-HSCs). Particularly, the present invention provides means and methods for genetically modifying HSCs involving gene-editing reagents, such as TALE-nucleases, that specifically target a non-functional endogenous RAG1 locus, comprising at least one mutation causing Severe Combined Immunodeficiency (SCID). As result, engineered RAG1-edited HSCs are provided, comprising an exogenous sequence comprising a nucleic acid sequence encoding a functional RAG1 protein, which is integrated in said HSCs' genome into a non-functional endogenous RAG1 locus, thereby restoring the normal cellular phenotype by enabling the expression of a functional RAG1 protein.

The present invention can be further summarized by the following items:
1. An engineered RAG1-edited Haematopoietic Stem Cell (HSC) comprising an exogenous sequence comprising, from its 5′ to 3′ ends, the nucleic acid sequence SEQ ID NO: 12, a nucleic acid sequence encoding a functional RAG1 protein, and a polyA signal, wherein said exogenous sequence is integrated in said HSC's genome into Intron 1 (SEQ ID NO: 28) of a non-functional endogenous RAG1 locus.
2. The engineered HSC according to item 1, wherein said nucleic acid sequence encoding a RAG1 protein is a codon optimized nucleic acid sequence.
3. The engineered HSC according to item 2, wherein said codon optimized nucleic acid sequence of RAG1 comprises the nucleic acid sequence SEQ ID NO: 19, SEQ ID NO: 20, SEQ ID NO: 68 or SEQ ID NO: 71; preferably SEQ ID NO: 68 or SEQ ID NO: 71.
4. The engineered HSC according to any one of items 1 to 3, wherein said polyA signal comprises the nucleic acid sequence SEQ ID NO: 27.
5. The engineered HSC according to any one of items 1 to 4, wherein said exogenous sequence further comprises a 5′ UTR of SEQ ID NO: 29 placed between said nucleic acid sequence SEQ ID NO: 12 and said nucleic acid sequence encoding a functional RAG1 protein.
6. The engineered HSC according to any one of items 1 to 5, wherein said exogenous sequence comprises the nucleic acid sequence SEQ ID NO: 23, SEQ ID NO: 25, SEQ ID NO: 69, or SEQ ID NO: 72; preferably SEQ ID NO: 69, or SEQ ID NO: 72.
7. A population of cells comprising engineered HSCs according to any one of items 1 to 6.
8. The population of cells according to item 7, comprising at least 1%, at least 5%, at least 10%, at least 15%, at least 20%, at least 25%, at least 30%, at least 35%, or at least 40% of said engineered RAG1-edited HSCs.
9. A population of cells according to item 7 or 8, comprising long-term repopulating HSCs.
10. The population of cells according to item 9, wherein at least 10%, at least 15%, at least 20%, at least 25%, or at least 30% of said long-term repopulating HSCs are engineered RAG1-edited HSCs.
11. A TALE-Nuclease heterodimer targeting intron 1 (SEQ ID NO: 28) of an endogenous RAG1 locus in an HSC.
12. A TALE-Nuclease heterodimer according to item 11, targeting the polynucleotide sequence of SEQ ID NO: 41.
13. The TALE-Nuclease heterodimer according to item 11 or 12, comprising a first monomer targeting the polynucleotide sequence of SEQ ID NO: 42, and a second monomer targeting the polynucleotide sequence of SEQ ID NO: 43.
14. The TALE-Nuclease heterodimer according to any one of items 11 to 13, comprising a first monomer comprising the amino acid sequence of SEQ ID NO: 4 or a variant thereof comprising an amino acid sequence having at least 80% sequence identity with SEQ ID NO: 4 and targeting the sequence of SEQ ID NO: 42 and a second monomer comprising the amino acid sequence of SEQ ID NO: 5 or a variant thereof comprising an amino acid sequence having at least 80% sequence identity with SEQ ID NO: 5 and targeting the sequence of SEQ ID NO: 43.
15. The TALE-Nuclease heterodimer according to any one of items 11 to 13, comprising a first monomer comprising the amino acid sequence of SEQ ID NO: 4 and a second monomer comprising the amino acid sequence of SEQ ID NO: 5.
16. A TALE-Nuclease heterodimer according to item 11, targeting the polynucleotide sequence of SEQ ID NO: 38.
17. The TALE-Nuclease heterodimer according to item 11 or 16, comprising a first monomer targeting the polynucleotide sequence of SEQ ID NO: 39, and a second monomer targeting the polynucleotide sequence of SEQ ID NO: 40.
18. The TALE-Nuclease heterodimer according to item 11, 16 or 17, comprising a first monomer comprising the amino acid sequence of SEQ ID NO: 2 or a variant thereof comprising an amino acid sequence having at least 80% sequence identity with SEQ ID NO: 2 and targeting the sequence of SEQ ID NO: 39 and a second monomer comprising the amino acid sequence of SEQ ID NO: 3 or a variant thereof comprising an amino acid sequence having at least 80% sequence identity with SEQ ID NO: 3 and targeting the sequence of SEQ ID NO: 40.
19. The TALE-Nuclease heterodimer according to item 11, 16, 17 or 18, comprising a first monomer comprising the amino acid sequence of SEQ ID NO: 2 and a second monomer comprising the amino acid sequence of SEQ ID NO: 3.
20. An isolated nucleic acid or vector encoding a TALE-Nuclease heterodimer according to any one of items 11 to 19.
21. The isolated nucleic acid according to item 20, which is a mRNA.
22. An isolated nucleic acid or a vector comprising an exogenous sequence as defined in any one of items 1 to 6, wherein said exogenous sequence is placed between a left homologous region at its 5′ end and a right homologous region at its 3′ end, homologous regions being relative to parts of intron 1 of SEQ ID NO: 28 of RAG1.
23. The vector according to item 22, which is a non-integrative viral vector, such as an AAV vector or IDLV vector.
24. The vector according to item 22 or 23, which is an AAV vector, such an AAV6 vector.
25. The isolated nucleic acid according to item 22, which is a single-stranded DNA.
26. A population of cells comprising HSCs comprising a TALE-Nuclease heterodimer according to any one of items 11 to 19, and comprising an exogenous sequence as defined in any one of items 1 to 6 and/or an isolated nucleic acid or vector according to any one of items 22 to 25.
27. The population according to item 26, comprising HSCs comprising a TALE-Nuclease heterodimer according to any one of items 12 to 15, and comprising an exogenous sequence as defined in any one of items 1 to 6 and/or an isolated nucleic acid or vector according to any one of items 22 to 25.
28. The population according to item 26, comprising HSCs comprising a TALE-Nuclease heterodimer according to any one of items 16 to 19, and comprising an exogenous sequence as defined in any one of items 1 to 6 and/or an isolated nucleic acid or vector according to any one of items 22 to 25.
29. A pharmaceutical composition comprising an engineered HSC according to any one of items 1 to 6, or a population of cells according to any one of items 7 to 10, and 26 to 28, and a pharmaceutically acceptable excipient and/or carrier.
30. An engineered HSC according to any one of items 1 to 6, a population of cells according to any one of items 7 to 10, and 26 to 28, or a pharmaceutical composition according to item
29, for use in the treatment of a Severe Combined Immunodeficiency (SCID) related to RAG1.
31. An engineered HSC according to any one of items 1 to 6, a population of cells according to any one of items 7 to 10, and 26 to 28, or a pharmaceutical composition according to item 29, for use in haematopoietic stem cell transplantation.
32. A method for treating a Severe Combined Immunodeficiency (SCID) related to RAG1 in a patient in need thereof, the method comprising administering an engineered HSC according to any one of items 1 to 6, a population of cells according to any one of items 7 to 10, and 26 to 28, or a pharmaceutical composition according to item 29, to said patient.
33. A method for haematopoietic stem cell transplantation in a patient in need thereof, the method comprising administering an engineered HSC according to any one of items 1 to 6, a population of cells according to any one of items 7 to 10, and 26 to 28, or a pharmaceutical composition according to item 29, to said patient.
34. A kit comprising:
   (i) at least one isolated nucleic acid or vector according to any one of items 22 to 25, and
   (ii) at least one isolated nucleic acid or vector encoding a TALE-nuclease heterodimer according to any one of items 11 to 19.
35. The kit according to item 34 comprising an isolated nucleic acid or vector comprising the nucleic acid sequence SEQ ID NO: 18, SEQ ID NO: 26, 70, or SEQ ID NO: 73, and an isolated nucleic acid or vector comprising a nucleic acid sequence encoding the amino acid sequences of SEQ ID NO: 4 and SEQ ID NO: 5.
36. Ex vivo use of the TALE-Nuclease heterodimer according to any one of items 11 to 19, or an isolated nucleic acid or vector encoding said TALE-Nuclease heterodimer, or a kit according to item 34 or 35, in integrating a functional RAG1 sequence in intron 1 of SEQ ID NO: 28 of a non-functional RAG1 endogenous locus of an HSC.
37. A method of preparing engineered RAG1-edited haematopoietic stem cells comprising introducing into the HSCs to be engineered:
   (i) at least one isolated nucleic acid or vector comprising an exogenous sequence as defined in any one of items 1 to 6, wherein said exogenous sequence is placed between a left homologous region at its 5′ end and a right homologous region at its 3′ end, homologous regions being relative to parts of intron 1 of SEQ ID NO: 28 of RAG1; and
   (ii) at least one isolated nucleic acid or vector coding for a sequence specific reagent inducing DNA cleavage that is capable of targeting and cleaving a sequence within intron 1 of SEQ ID NO: 28 of the endogenous RAG1 locus of said HSCs;
   whereby engineered RAG1 edited haematopoietic stem cells are obtained.
38. The method according to item 37, wherein said sequence specific reagent inducing DNA cleavage is the TALE-nuclease heterodimer according to any one of items 11 to 19.
39. The method according to item 37 or 38, wherein said sequence specific reagent inducing DNA cleavage is the TALE-nuclease heterodimer according to any one of items 12 to 15.
40. The method according to any one of items 37 to 39, wherein said nucleic acid or vector of (i) comprises an exogenous sequence comprising the nucleic acid sequence SEQ ID NO: 18, SEQ ID NO: 26, SEQ ID NO: 70, or SEQ ID NO: 73, and wherein said nucleic acid or vector of (ii) comprises a nucleic acid sequence encoding the amino acid sequences of SEQ ID NO: 4 and SEQ ID NO: 5.
41. The method according to any one of items 37 to 40, wherein in (i) said vector is a non-integrative viral vector such as an AAV or IDLV.
42. The method according to item 41, wherein said vector is an AAV vector, such as an AAV6 vector.
43. The method according to any one of items 37 to 40, wherein in (i) said isolated nucleic acid is a single-stranded DNA.
44. The method according to any one of items 37 to 43, wherein in (ii) said isolated nucleic acid is introduced into the HSCs as mRNA by electroporation.
45. The method according to any one of items 37 to 44, comprising introducing a dominant negative fragment of the p53 protein, such as GSE56 of SEQ ID NO: 10, and/or a p53-binding protein 1 (53BP1) inhibitor, such as the p53-binding protein 1 inhibitor of SEQ ID NO: 11, or a dominant-negative form of tumour suppressor p53-binding protein 1.
46. The method according to any one of items 37 to 45, comprising introducing a nucleic acid comprising a nucleic acid sequence encoding a dominant negative p53 fragment, such as GSE56 of SEQ ID NO: 10, and/or a nucleic acid sequence comprising a nucleic acid sequence encoding a p53-binding protein 1 inhibitor, such as the p53-binding protein 1 inhibitor of SEQ ID NO: 11 or a dominant-negative form of tumour suppressor p53-binding protein 1.
47. The method according to any one of items 37 to 46, wherein said HSCs are isolated from a tissue sample from a human donor.
48. The method according to item 47, wherein said human donor is a human patient suffering from Severe Combined Immunodeficiency (SCID) related to RAG1.
49. The method according to item 47 or 48, wherein the tissue sample is a peripheral blood sample.
50. The method according to any one of items 47 to 49, wherein the tissue sample is obtained from a human donor who has been administered with at least one HSC mobilizing agent prior to obtaining the tissue sample.
51. The method according to item 50, wherein the HSC mobilizing agent is G-CSF and/or plerixafor.
52. The engineered RAG1-edited haematopoietic stem cells obtainable by the method according to any one of items 37 to 51.
53. The engineered RAG1-edited haematopoietic stem cells obtainable by the method according to any one of items 37 to 51, for use in treatment of Severe Combined Immunodeficiency (SCID) related to RAG1.
54. The engineered RAG1-edited haematopoietic stem cells obtainable by the method according to any one of items 37 to 51, for use in treatment of Severe Combined Immunodeficiency (SCID) related to RAG1 in a patient who was the donor of the HSCs to be engineered.
55. The engineered RAG1-edited haematopoietic stem cells obtainable by the method according to any one of items 37 to 51, for use in haematopoietic stem cells transplantation.

### BRIEF DESCRIPTION OF THE DRAWINGS

FIG. 1: SCID-causing RAG1 mutations. a) Schematic representation of the recombination-activating gene 1 (RAG1) protein with the various mutations according to the clinical presentation. RAG1 catalytic residues and zinc-binding residues are indicated by stars, respectively. b) Number of RAG1 missense mutations in the various domains. c) Frequency of RAG1 mutations calculated by dividing the number of mutations in a given region by the number of amino acids in that region. Figure taken from [5]. BI, basic I domain; BIIa/b, basic IIa/b domain; BIII, basic Ill domain; CTD, carboxy-terminal domain; DDBD, dimerization and DNA-binding domain; NBD, nonamer-binding domain; PHD, plant homeodomain; preR, pre-RNase H; RNH, catalytic RNase H.

FIG. 2: Gene editing strategy. (A) Schematic outline of the RAG1 gene editing strategy. TALEN-mediated cleavage within intron 1 of RAG1 stimulates targeted integration of a donor template containing a corrective codon-optimized copy of RAG1 delivered by AAV6 transduction. Five different donor templates (AAV #1-5 of SEQ ID Nos: 14-18, respectively) were designed and tested, containing 300 bp left and right homology arms (HA) of SEQ ID NO: 21 and SEQ ID NO: 22, respectively, a polyadenylation signal derived from the bovine growth hormone locus (bGH pA), coRAG1, and different SA/5′UTR sequences. All donor templates (AAV #1-5) contained a 5′UTR as defined in SEQ ID 29. The donor templates AAV #1-4 contained a part of the RAG1 intron 1 (50-140 bp of exon 2 proximal sequence), including the natural Splice Acceptor (SA) site, followed by said 5′UTR, whereas donor template AAV #5 contained a short Artificial Splice Acceptor (SA) site followed by said 5′UTR. (B) Expression of coRAG1 in Jurkat cells edited with TALEN-6 and AAV #1-5. Shown is fold-expression relative to AAV #1. 5′/3′UTR=5/3′ untranslated region, coRAG1=codon-optimized RAG1.

FIG. 3: Characterization of the TALENs targeting RAG1 intron 1. (A) Localization of different TALENs targeting the 3′-terminal region of intron 1 of the RAG1 gene. (B) Activity of TALEN-2 evaluated by T7 Endonuclease I (T7EI) assay. Arrows show cut PCR products. (C) Activity (indels %) of TALEN-6, TALEN-A and TALEN-B assessed by Next Generation Sequencing (NGS).

FIG. 4: Knock-in of coRAG1 in primary human HSCs. Quantification of coRAG1 knock-in in CD34+ HSCs after cleavage mediated by TALEN-2 (A-B) or TALEN-6 (C-D) in the presence or absence of 53BP1 inhibitor and/or GSE56. Knock-in frequencies of AAV #5 was determined by 3′ and 5′ in-out ddPCR on bulk population (bulk) or on sorted Long Term HSCs population (LT-HSC) and on sorted Short Term HSC population (ST-HSC) (D).

FIG. 5: Differentiation potential of edited cells. (A) Untreated HSCs and HSCs edited with TALEN-6 and AAV #5 were subjected to CFU assay. Per condition, two plates were seeded, and colony numbers and types evaluated independently by two individuals. Shown are the percentages of the various CFU colony types. BFU-E=Burst-forming unit-erythroid, CFU-E=Colony-forming unit-erythroid, CFU-G=Burst-forming unit-granulocyte, CFU-GEMM=Burst-forming unit-granulocyte/erythrocyte/macro-phage/megakaryocyte, CFU-GM=Burst-forming unit-granulocyte/macrophage, CFU-M=Burst-forming unit-macrophage. (B) Quantification of mono- and bi-allelic knock-in in individual colonies collected from CFU assay plates. Twenty colonies (ten each from HSCs edited with TALEN plus AAV #5 and treated with and without 53BP1 inhibitor, respectively) were collected from CFU assay plates after two weeks. Mono-/bi-allelic integration was determined by PCR using primers that target the 3′ and 5′ junctions.

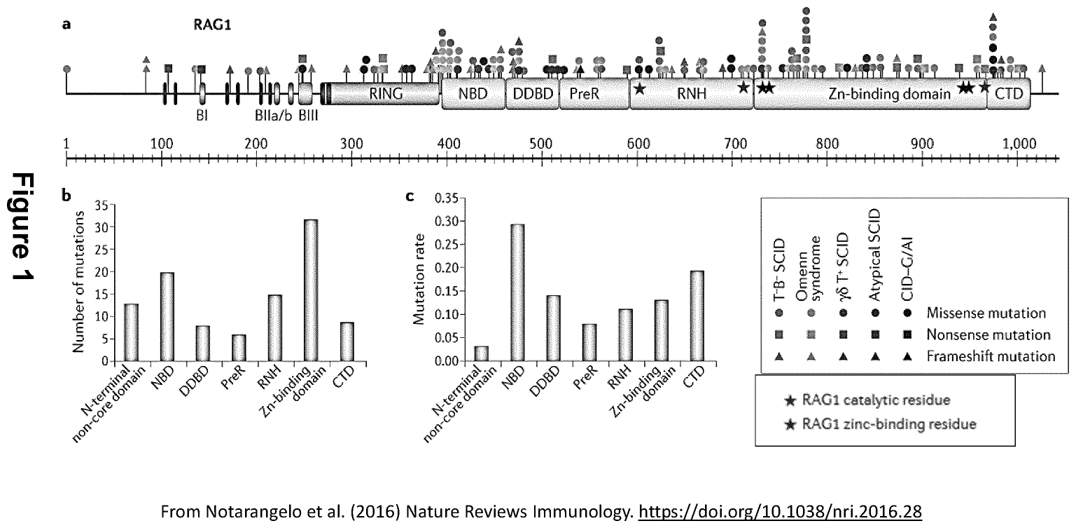

### DETAILED DESCRIPTION OF THE INVENTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, genetics, transgenic biology, microbiology, recombinant DNA, and immunology, which are within the skill of the art. Such techniques are explained fully in the literature. See, for example, Current Protocols in Molecular Biology [Frederick M. AUSUBEL (2000) Wiley and son Inc, Library of Congress, USA; Molecular Cloning: A Laboratory Manual, Third Edition] [Sambrook et al (2001) Cold Spring Harbor, New York: Cold Spring Harbor Laboratory Press]; Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Cells and Cell Populations of the Present Invention

As noted above, the present invention is an improved gene therapy approach to treat Severe Combined Immunodeficiency (SCID) related to RAG1, allowing the correction of RAG1 deficiency in HSCs, notably in long-term repopulating (HSCs). Particularly, the present invention provides means and methods for genetically modifying HSCs involving gene-editing reagents, such as TALE-nucleases, that specifically target a non-functional endogenous RAG1 locus, comprising at least one mutation causing Severe Combined Immunodeficiency (SCID). As result, engineered RAG1-edited HSCs are provided, comprising an exogenous sequence comprising a nucleic acid sequence encoding a functional RAG1 protein, which is integrated in said HSCs' genome into a non-functional endogenous RAG1 locus, thereby restoring the normal cellular phenotype by enabling the expression of a functional RAG1 protein.

Given that Severe Combined Immunodeficiency (SCID) is caused by one or more mutations within Exon 2 of the RAG1 gene which encodes the entire RAG1 protein, the present invention particularly aims at replacing the defective RAG1 gene sequence downstream of Intron 1 of the endogenous RAG1 locus in HSCs, notably HSCs originating from a patient suffering from SCID, by a corrective RAG1 coding sequence. Moreover, as shown in the examples, the present inventors have surprisingly found that RAG1 expression is greatly improved when an artificial splice site, notably the artificial splice site of SEQ ID NO: 12, is placed 5′ of the RAG1 coding sequence in the exogenous sequence compared to constructs where a natural splice site is used instead.

Thus, in a general aspect, the present invention is drawn to an engineered RAG1-edited Haematopoietic Stem Cell (HSC) comprising an exogenous sequence comprising, from its 5′ to 3′ ends, the nucleic acid sequence of SEQ ID NO: 12, a nucleic acid sequence encoding a functional RAG1 protein, and a polyA signal, wherein said exogenous sequence is integrated in said HSC's genome into intron 1 (SEQ ID NO: 28) of a non-functional endogenous RAG1 locus.

With “functional RAG1 protein” it is meant the RAG1 protein of SEQ ID NO: 1. An exemplary, non-limiting, coding sequence is set forth in SEQ ID NO: 13.

With “non-functional endogenous RAG1 locus” it is meant at least one allele of an endogenous RAG1 gene, which contains one or more mutations causing a disease state such as Severe Combined Immunodeficiency (SCID). To date, 150 RAG1 mutations have been identified in SCID patients, including 103 missense mutations, 18 nonsense mutations and 29 frameshift mutations [5].

Generally, once integrated in the endogenous RAG1 locus, said exogenous sequence allows the expression of the RAG1 protein of SEQ ID NO: 1.

The engineered HSC may be a primary cell. Primary cells are generally used in cell therapy as they are deemed more functional and less tumorigenic. In general, primary HCS cells can be obtained from a patient suffering from SCID through a variety of methods known in the art. For example, primary HSCs can be taken from bone marrow, and more particularly from the pelvis, at the iliac crest, using a needle or syringe. Alternatively, HSCs may be harvested from the circulating peripheral blood, while blood donors are injected with a HSC mobilizing agent, such as chemokine (C-X-C motif) receptor 4 (CXCR4) antagonists, such as AMD3100 (also known as Plerixafor and MOZOBIL (Genzyme, Boston, Mass.)), granulocyte-colony stimulating factor (G-CSF), and chemokine (C-X-C motif) ligand 2 (CXCL2, also referred to as GROβ), which induces cells to leave the bone marrow and circulate in the blood vessels. HSCs may also be harvested from cord blood.

According to some embodiments, the engineered HSC is a mammalian HSC, preferably is a human HSC.

According to some embodiments, the engineered HSC is a long-term repopulating HSC (LT-HSC).

According to some embodiments, the engineered HSC is a short-term repopulating HSC (ST-HSC).

According to some embodiments, the engineered HSC is at least CD34+.

According to some embodiments, the engineered HSC is at least CD34+, CD90+, and CD133+.

According to some embodiments, the engineered HSC is at least CD34+, CD38−, CD45RA−, CD90+, and CD133+.

According to some embodiments, the engineered HSC is at least CD34+, CD38−, and CD45RA−.

According to some embodiments, the engineered HSC is at least CD34+, CD38−, CD45RA−, CD90−, and CD133−.

In order to improve the expression of the functional RAG1 polypeptide and to avoid homologous recombination with the endogenous RAG1 coding sequence, said nucleic acid sequence encoding a RAG1 protein may be codon optimized. Thus, according to some embodiments, said nucleic acid sequence encoding a RAG1 protein is a codon optimized nucleic acid sequence.

With “codon optimized” it is meant that the polynucleotide sequence has been adapted for expression in the cells of a given vertebrate, such as a human or other mammal, by replacing at least one, or more than one, or a significant number, of codons with one or more codons that are more frequently used in the genes of that vertebrate. Accordingly, the nucleic acid sequence encoding a RAG1 protein can be tailored for optimal gene expression in a given organism based on codon optimization. Codon optimization can be done based on established codon usage tables, for example, at the “Codon Usage Database” available at http://www.kazusa.or.jp/codon/(hosted by Kazusa DNA Research Institute, Japan), or other kind of computer algorithms. A non-limiting example is the OptimumGene PSO algorithm from GenScript® which takes into consideration a variety of critical factors involved in different stages of protein expression, such as codon adaptability, mRNA structure, and various cis-elements in transcription and translation. By utilizing codon usage tables or other kind of computer algorithms, one of ordinary skill in the art can apply the frequencies to any given polypeptide sequence, and produce a nucleic acid fragment of a codon-optimized coding region which encodes the polypeptide, but which uses codons optimal for a given organism.

Preferably, codon optimization also includes the removal of any cryptic splicing site as well as any microRNA target site and/or a sequence which would generate a mRNA secondary structure impeding translation. Thus, according to some embodiments, said codon optimized nucleic acid sequence has been further optimized to remove at least one cryptic splicing site and/or to remove at least one microRNA target site and/or a sequence which would generate a mRNA secondary structure impeding translation. Computer algorithms allowing the prediction of cryptic splicing sites are well known to the skilled person, and include, for example, the splice prediction tool available at http://wangcomputing.com/assp/. Similarly, one of ordinary skill in the art can use established computer algorithms, such as the miRNA target prediction tool available at http://mirdb.org/, to identify miRNA target sites.

According to some embodiments, said codon optimized nucleic acid sequence of RAG1 comprises the nucleic acid sequence of SEQ ID NO: 19 or SEQ ID NO: 20.

According to some embodiments, said codon optimized nucleic acid sequence of RAG1 comprises the nucleic acid sequence of SEQ ID NO: 19.

According to some embodiments, said codon optimized nucleic acid sequence of RAG1 comprises the nucleic acid sequence of SEQ ID NO: 20.

According to further embodiments, said codon optimized nucleic acid sequence of RAG1 comprises the nucleic acid sequence of SEQ ID NO: 68 or SEQ ID NO: 71.

Further, in order to facilitate the nuclear export, translation and stability of mRNA, the exogenous sequence comprises a polyA signal. Non-limiting examples of polyA signals include polyA signals from SV40, hGH (human Growth Hormone), bGH (bovine Growth Hormone), and rbGlob (rabbit beta-globin). According to some embodiments, said polyA signal comprises the nucleic acid sequence of SEQ ID NO: 27.

Further, in order to allow regulation of translation of the coding sequence from mRNA, said exogenous sequence may further comprise a 5′ untranslated region (5′ UTR) (also known as a leader sequence, transcript leader, or leader RNA) placed between said nucleic acid sequence of SEQ ID NO: 12 and said nucleic acid sequence encoding a functional RAG1 protein. A non-limiting example of such 5′UTR is set forth in SEQ ID NO: 29.

According to some embodiments, said exogenous sequence comprises SEQ ID NO: 23.

According to some embodiments, said exogenous sequence comprises SEQ ID NO: 25.

According to some embodiments, said exogenous sequence comprises SEQ ID NO: 69.

According to some embodiments, said exogenous sequence comprises SEQ ID NO: 72.

The present invention further provides a population of cells comprising engineered RAG1-edited HSCs according to the invention.

Preferably, said population of cells comprises at least about 1%, such as at least 10%, of said engineered RAG1-edited HSCs.

According to some embodiments, at least 20%, such as at least 30% or at least 40%, of the total cells of the population of cells are said engineered RAG1-edited HSCs.

According to some embodiments, at least 50%, such as at least 60% or at least 70%, of the total cells of the population of cells are said engineered RAG1-edited HSCs.

According to some embodiments, at least 80%, such as at least 90% or at least 95%, of the total cells of the population of cells are said engineered RAG1-edited HSCs.

The population of cells according to the present invention may comprise long-term repopulating HSCs (LT-HSCs). According to some embodiments, at least 10%, such as at least 15%, at least 20%, at least 25% or at least 30%, of said LT-HSCs are engineered RAG1 edited HSCs of the present invention.

The present invention further provides engineered RAG1 edited HSCs as well as populations of engineered RAG1 edited HSCs obtainable by any of the production methods disclosed herein.

### Means and Method for Gene Editing According to the Invention

The present invention further provides means and methods for genetically modifying HSCs involving gene editing reagents that specifically target a non-functional endogenous RAG1 gene, comprising at least one mutation causing Severe Combined Immunodeficiency (SCID), thereby allowing the restoration of the normal cellular phenotype. Targeted (i.e. site-directed) integration to achieve gene function is suitably done by using sequence-specific reagents inducing DNA cleavage, such as a rare-cutting endonuclease or nickase, and exogenous polynucleotide donor templates bearing homology to the target site and comprising the corrective sequence. Targeted integration can also been achieved using sequence-specific reagents inducing transposition such as described by Owns et al. [6], Voigt et al. [7] or Bhatt and Chalmers [8].

The present invention thus provides sequence specific reagents inducing DNA cleavage that are capable of targeting and cleaving a sequence within Intron 1 of the endogenous RAG1 locus.

Non-limiting examples of a “sequence-specific reagent inducing DNA cleavage” according to the invention include reagents that have nickase or endonuclease activity. The sequence-specific reagent can be a chimeric polypeptide comprising a DNA binding domain and another domain displaying catalytic activity. Such catalytic activity can be for instance a nuclease to perform gene inactivation, or nickase or double nickase to preferentially perform gene insertion by creating cohesive ends to facilitate gene integration by homologous recombination, or to perform base editing as described in Komor et al. [9].

In general, the sequence specific reagents of the present invention have the ability to recognize and bind a “target sequence” within Intron 1 (SEQ ID NO: 28) of an endogenous RAG1 locus in an HSC. The “target sequence” which is recognized and bound by the sequence specific reagents is usually selected to be rare or unique in Intron 1, as can be determined using software and data available from human genome databases, such as http://www.ensembl.org/index.html. Such “target sequence” is preferably one set forth in SEQ ID NO: 41 (target region of TALEN-6) or SEQ ID NO: 38 (target region of TALEN-2).

Thus, according to the present invention, sequence-specific reagents inducing DNA cleavage that are capable of targeting and cleaving a sequence as set forth in SEQ ID NO: 41 or SEQ ID NO: 38 are provided.

According to some embodiments, the sequence-specific reagent inducing DNA cleavage is a rare-cutting endonuclease. “Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to some embodiments, said rare-cutting endonuclease is an “engineered” or “programmable” rare-cutting endonuclease, such as a homing endonuclease as described for instance by Arnould S., et al. (WO2004067736), a zing finger nuclease (ZFN) as described, for instance, by Urnov F., et al. [10], a TALE-nuclease as described, for instance, by Mussolino et al. [11], or a MegaTAL nuclease as described, for instance by Boissel et al. [12]. Due to their higher specificity, TALE-nucleases have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3′” or “reverse”) as reported for instance by Mussolino et al. [13]. Thus, according to some preferred embodiments, said rare-cutting endonuclease is a TALE-nuclease.

The present invention thus provides a TALE-Nuclease heterodimer targeting Intron 1 of an endogenous RAG1 locus in an HSC.

Particularly, the present invention provides a TALE-Nuclease heterodimer targeting the polynucleotide sequence of SEQ ID NO: 41 (target region of TALEN-6).

According to some embodiments, said TALE-Nuclease heterodimer comprises a first monomer targeting the polynucleotide sequence of SEQ ID NO: 42, and a second monomer targeting the polynucleotide sequence of SEQ ID NO: 43.

According to some embodiments, said TALE-Nuclease heterodimer comprises a first monomer comprising the amino acid sequence of SEQ ID NO: 4 or a variant thereof comprising an amino acid sequence having at least 80% sequence identity with SEQ ID NO: 4 and targeting the sequence 42 and a second monomer comprising the amino acid sequence of SEQ ID NO: 5 or a variant thereof comprising an amino acid sequence having at least 80% sequence identity with SEQ ID NO: 5 and targeting the sequence 43.

According to some embodiments, said TALE-Nuclease heterodimer comprises a first monomer comprising the amino acid sequence of SEQ ID NO: 4 and a second monomer comprising the amino acid sequence SEQ ID NO: 5.

These monomers and variants thereof are designed to bind their respective target polynucleotide sequences SEQ ID NO: 42 and SEQ ID NO: 43 and comprise the RVD sequence NI-NG-NN-NI-NG-HD-NI-NN-HD-NI-HD-HD-NG-NI-NI-NG and RVD sequence NI-NG-NN-NG-NI-NG-NG-NI-NI-NI-NG-HD-NG-NG-HD-NG, respectively. The cleavage occurs within the endogenous RAG1 sequence, between the target sequences.

The present invention also provides a TALE-Nuclease heterodimer targeting the polynucleotide sequence of SEQ ID NO: 38 (target region of TALEN-2).

According to some embodiments, said TALE-Nuclease heterodimer comprises a first monomer targeting the polynucleotide sequence of SEQ ID NO: 39, and a second monomer targeting the polynucleotide sequence of SEQ ID NO: 40.

According to some embodiments, said TALE-Nuclease heterodimer comprises a first monomer comprising the amino acid sequence of SEQ ID NO: 2 or a variant thereof comprising an amino acid sequence having at least 80% sequence identity with SEQ ID NO: 2 and targeting the sequence 39 and a second monomer comprising the amino acid sequence of SEQ ID NO: 3 or a variant thereof comprising an amino acid sequence having at least 80% sequence identity with SEQ ID NO: 3 and targeting the sequence 40.

According to some embodiments, said TALE-Nuclease heterodimer comprises a first monomer comprising the amino acid sequence of SEQ ID NO: 2 and a second monomer comprising the amino acid sequence of SEQ ID NO: 3.

These monomers and variants thereof are designed to bind their respective target polynucleotide sequences SEQ ID NO: 39 and SEQ ID NO: 40 and comprise the RVD sequence HD-NG-NI-NG-NN-NI-NG-HD-NI-NN-HD-NI-HD-HD-NG-NG and the RVD sequence NN-NG-NI-NG-NG-NI-NI-NI-NG-HD-NG-NG-HD-NG-NI-NG, respectively. The cleavage occurs within the endogenous RAG1 sequence, between the target sequences.

According to some embodiments, the rare-cutting endonuclease is an RNA guided endonuclease, such as Cas9 or Cpf1, to be used in conjunction with a RNA-guide as per, inter alia, the teaching by Doudna, J. et al., [14] and Zetsche, B. et al. [15]. The RNA-guide is designed to hybridize a target sequence.

According to some embodiments, the sequence-specific reagent inducing DNA cleavage is a nickase.

The present invention further provides an isolated nucleic acid encoding the sequence-specific reagent inducing DNA cleavage of the present invention.

According to some embodiments, said isolated nucleic acid is a mRNA.

The present invention further provides a vector, such as a viral vector, and more specifically a non-integrative viral vector such as an AAV or IDLV vector, comprising a polynucleotide sequence encoding the sequence-specific reagent inducing DNA cleavage of the present invention.

The present invention also provides the ex vivo use of the sequence specific reagent inducing DNA cleavage of the invention, the isolated nucleic acid of the invention encoding same or the vector encoding same in gene editing HSCs, notably HSCs of a patient suffering from SCID related to RAG1.

The present invention further provides an exogenous sequence as defined above in the form of an isolated nucleic acid or vector comprising same. The isolated nucleic acid or vector will function as a polynucleotide donor template.

In order to facilitate targeted (i.e. site-directed) integration of the exogenous sequence via homologous recombination, said exogenous sequence is placed between a left and/or a right homology sequence having at least 80% sequence identity with a part of Intron 1 of the endogenous RAG1 locus. Specifically, the exogenous sequence is placed between a left homologous region at its 5′ end and a right homologous region at its 3′ end, homologous regions being relative to parts of Intron 1 of SEQ ID NO: 28 of RAG1.

In order to facilitate targeted (i.e. site-directed) integration of the exogenous sequence via NHEJ repair mechanism at the cleaved locus, said exogenous sequence may comprise microhomologies, i.e. short homologous DNA sequences.

The present invention further provides an isolated nucleic acid comprising said exogenous sequence. Said isolated nucleic acid comprising said exogenous sequence can be provided either as a double- or single-stranded polynucleotide that can be electroporated into the cells, such as a single-stranded DNA (“ssDNA”) and a double-stranded DNA (“dsDNA”), or as part of a viral vector (e.g., AAV) through viral transduction. This isolated nucleic acid comprising said exogenous sequence, functioning as a polynucleotide donor template, is introduced into the cells by methods well known in the art.

The present invention further provides a vector comprising said exogenous sequence, such as a viral vector, and more specifically a non-integrative viral vector such as an AAV or IDLV vector, comprising said exogenous sequence. Thus, according to some embodiments, the vector is an AAV vector, preferably an AAV6 vector. AAV vectors, and especially AAV6, are particularly suited for transduction of polynucleotide donor templates into cells and to perform integration by homologous recombination directed by rare-cutting endonucleases as described for instance by Sather, B. D. et al. [16].

The present invention further provides a single-stranded nucleic acid comprising said exogenous sequence, such as a single-stranded DNA (“ssDNA”).

The present invention also provides a double-stranded nucleic acid comprising said exogenous sequence, such as a double-stranded DNA (“dsDNA”).

In the present invention, single-stranded DNAs and double-stranded DNAs can be advantageous over viral vectors in several respects. For example, single-stranded DNAs and double-stranded DNAs do not contain vector-specific sequences such as LTR and ITR, and may avoid contamination by undesired plasmid sequences during production process. According to some embodiments, said single-stranded DNA or double-stranded DNA can comprise protection of DNA ends or specific structures (such as hairpin, loop) that will protect the donor template from degradation. They can also comprise modifications such as modified sugar moiety or modified inter-nucleoside linkage as described in WO2012065143. In some embodiments, dsDNA ends can be covalently closed. The sequences of the ssDNAs and dsDNAs can be optimized more easily as they are usually synthetized in vitro, thereby allowing the optional incorporation of modified bases (e.g., methylation, biotinylation . . . ). In some embodiments, the sequences of the ssDNAs and dsDNAs can incorporate the sequence of a site-specific nuclease such as one described in the present invention. Furthermore, ssDNAs and dsDNAs may be cheaper to produce under good manufacturing practices (GMP) than viral vectors which are produced in host cells. In terms of specificity, ssDNAs are regarded as allowing more specific and stable genomic integration as resorting mainly to rad51 independent mechanism rather than classic homologous recombination (rad51 dependent). Such integration can be further promoted by treating the cells with specific molecules, such as inhibitors of 53BP1 and/or GSE56; and/or siRNA such as rad51 siRNA; and/or Rad59mRNA or helicases mRNAs such as Srs2, UvrD, PcrA, Rep or FBH1.

The present invention also provides the ex vivo use of an isolated nucleic acid or vector of the invention, comprising the exogenous sequence as defined herein, in gene editing HSCs, notably HSCs of a patient suffering from SCID related to RAG1.

The present invention also provides the ex vivo use of the sequence specific reagent inducing DNA cleavage of the invention, the polynucleotide of the invention encoding same or the vector encoding same in combination with an isolated nucleic acid or vector of the invention, comprising the exogenous sequence as defined herein, for use in gene editing HSCs, notably HSCs of a patient suffering from SCID related to RAG1.

The present invention further provides a kit comprising:
(i) at least one isolated nucleic acid or vector comprising an exogenous sequence as defined herein, and
(ii) at least one isolated nucleic acid or vector encoding the sequence-specific reagent inducing DNA cleavage of the present invention.

According to some embodiments, said kit comprises an isolated nucleic acid or vector comprising the nucleic acid sequence SEQ ID NO: 18, SEQ ID NO: 26, SEQ ID NO: 70, or SEQ ID NO: 73, and an isolated nucleic acid or vector comprising a nucleic acid sequence encoding the amino acid sequences of SEQ ID NO: 4 and SEQ ID NO: 5.

According to some embodiments, said kit comprises an isolated nucleic acid or vector comprising the nucleic acid sequence SEQ ID NO: 18, SEQ ID NO: 26, SEQ ID NO: 70, or SEQ ID NO: 73, and an isolated nucleic acid or vector comprising a nucleic acid sequence encoding the amino acid sequences of SEQ ID NO: 2 and SEQ ID NO: 3.

The present invention also provides the ex vivo use of the kits of the invention, in gene editing HSCs, notably HSCs of a patient suffering from SCID related to RAG1.

The present invention further provides a method of preparing engineered RAG1 edited haematopoietic stem cells comprising introducing into the HSCs to be engineered:
(i) at least one isolated nucleic acid or vector comprising an exogenous sequence as defined herein, wherein said exogenous sequence is placed between a left homologous region at its 5′ end and a right homologous region at its 3′ end, homologous regions being relative to parts of intron 1 of SEQ ID NO: 28 of RAG1; and
(ii) at least one isolated nucleic acid or vector coding for a sequence specific reagent inducing DNA cleavage of the present invention;
whereby engineered RAG1 edited haematopoietic stem cells are obtained.

The method has been particularly designed to obtain engineered RAG1-edited HSCs for the treatment of a patient suffering from SCID related to RAG1 by gene therapy, more particularly by integrating a corrective coding sequence for RAG1 at a non-functional endogenous RAG1 locus using the exogenous sequence and the sequence-specific reagent inducing DNA cleavage described herein.

The method is preferably practiced ex vivo to obtain stably engineered RAG1-edited HSCs. The resulting engineered HSCs can be then engrafted into a patient in need thereof for a long term in-vivo production of engineered cells that will comprise said exogenous sequence as detailed herein.

According to some embodiments, said nucleic acid or vector of (i) comprises an exogenous sequence comprising the nucleic acid sequence SEQ ID NO: 18, SEQ ID NO: 26, SEQ ID NO: 70, or SEQ ID NO: 73, and wherein said nucleic acid or vector of (ii) comprises a nucleic acid sequence encoding the amino acid sequences of SEQ ID NO: 4 and SEQ ID NO: 5.

According to some embodiments, said nucleic acid or vector of (i) comprises an exogenous sequence comprising the nucleic acid sequence SEQ ID NO: 18, SEQ ID NO: 26, SEQ ID NO: 70, or SEQ ID NO: 73, and wherein said nucleic acid or vector of (ii) comprises a nucleic acid sequence encoding the amino acid sequences of SEQ ID NO: 2 and SEQ ID NO: 3.

According to some embodiments, the sequence-specific reagent inducing DNA cleavage, such as a rare-cutting endonuclease, is transiently expressed or delivered in the cells, meaning that said reagent is not supposed to integrate into the genome or persist over a long period of time, such as would be the case of RNA, more particularly mRNA, proteins or complexes mixing proteins and nucleic acids (eg: Ribonucleoproteins). Preferably, the sequence-specific reagent inducing DNA cleavage, such as a rare-cutting endonuclease, is introduced into the cell in the form of a nucleic acid molecule, such as a DNA or RNA molecule, preferably mRNA molecule, encoding said sequence-specific reagent, and will be expressed by the transfected cell. A sequence-specific reagent inducing DNA cleavage, such as a rare-cutting endonuclease, under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

According to some embodiments, said sequence-specific reagent inducing DNA cleavage is introduced into said HSCs via a vector.

According to some embodiments, said sequence-specific reagent inducing DNA is introduced into said HSCs as mRNA by electroporation.

According to some embodiments, said sequence-specific reagent inducing DNA cleavage is introduced into said HSCs via nanoparticles, preferably nanoparticles which are coated with ligands, such as antibodies, having a specific affinity towards a HSC surface protein, such as CD105 (Uniprot #P17813). Preferred nanoparticles are biodegradable polymeric nanoparticles in which the sequence specific reagent under polynucleotide form is complexed with a polymer of polybeta amino ester and coated with polyglutamic acid (PGA).

According to some embodiments, methods of non-viral delivery of the exogenous sequence and/or the sequence-specific reagent inducing DNA cleavage can be used such as electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery.

According to some embodiments, electroporation steps can be used to transfect cells. In general, electroporation steps that are used to transfect cells are typically performed in closed chambers comprising parallel plate electrodes producing a pulse electric field between said parallel plate electrodes greater than 100 volts/cm and less than 5,000 volts/cm, substantially uniform throughout the treatment volume such as described in WO/2004/083379, especially from page 23, line 25 to page 29, line 11. One such electroporation chamber preferably has a geometric factor (cm-1) defined by the quotient of the electrode gap squared (cm2) divided by the chamber volume (cm3), wherein the geometric factor is less than or equal to 0.1 cm-1, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

The method may further involve the use of one or more polypeptides that enhances homologous recombination and/or viability of HSCs, such as a dominant negative p53 fragment, such as GSE56, and/or a p53-binding protein 1 inhibitor or a dominant-negative form of tumour protein p53-binding protein.

According to some embodiments, the method further comprises introducing a dominant negative p53 fragment, such as GSE56 of SEQ ID NO: 10, and/or a p53-binding protein 1 inhibitor, such as the p53-binding protein 1 inhibitor of SEQ ID NO: 11 or a dominant-negative form of tumour protein p53-binding protein 1.

According to some embodiments, the method further comprises introducing a nucleic acid comprising a nucleic acid sequence encoding a dominant negative p53 fragment, such as GSE56 of SEQ ID NO: 10, and/or a nucleic acid sequence comprising a nucleic acid sequence encoding a p53-binding protein 1 inhibitor, such as the p53-binding protein 1 inhibitor of SEQ ID NO: 11 or a dominant-negative form of tumour protein p53-binding protein 1.

According to some embodiments, said HSCs are isolated from a tissue sample from a human donor, such as a human patient suffering from Severe Combined Immunodeficiency (SCID) related to RAG1.

For example, said human patient suffering from SCID related to RAG1 can have at least one RAG1 mutation selected from the group consisting of R314W, C328Y, C358Y, K391E, R394Q, R394W, R396C, R396H, R396L, S401P, T403P, R404Q, R410Q, R410W, L411P, D429G, V433M, M435V, A444V, L454Q, R474C, L506F, L514R, G516A, W522C, D539V, R559S, R561H, H612R, R624H, R699Q/W, E722K, Y728H, C730F, L732P, R737H, R764P, R764H, E770K, R778Q, R778W, P786L, R841W, W896R, Y912C, 1956T, F974L, R975Q, R975W, Q981P and K992E.

According to some embodiments, said tissue sample is a peripheral blood sample.

According to some embodiments, said tissue sample is obtained from a human donor who has been administered with at least one HSC mobilizing agent, such as G-CSF and/or plerixafor, prior to obtaining the tissue sample.

The present invention further provides a population of cells comprising HSCs comprising a sequence specific reagent inducing DNA cleavage of the present invention and an exogenous sequence as defined herein.

According to some embodiments, said population comprises HSCs comprising a TALE-Nuclease heterodimer according to the present invention and an exogenous sequence as defined herein.

### Activation and Gene Editing of HSCs

For mobilised peripheral blood (MPB) leukapheresis, CD34+ cells are generally processed and enriched using immunomagnetic beads such as CliniMACS. Purified CD34+ cells are seeded on culture bags at 1×10⁶ cells/ml in serum-free medium in the presence of cell culture grade Stem Cell Factor (SCF), preferably 300 ng/ml, preferably with FMS-like tyrosine kinase 3 ligand (FLT3L) 300 ng/ml, and Thrombopoietin (TPO), preferably around 100 ng/ml and further interleukine IL-3, preferably more than 60 ng/ml (all from CellGenix) during between preferably 12 and 24 hours before being transferred to an electroporation buffer comprising mRNA encoding the sequence specific reagent. Upon electroporation, the cells are preferably cryopreserved.

### Therapeutic Methods, Compositions and Uses of the Invention

The present invention described above allows producing engineered RAG1-edited HSCs or populations comprising such engineered cells in which the initially defective endogenous RAG1 gene sequence causing SCID has been replaced by, and/or integrated, a corrective RAG1 sequence, thereby restoring the normal cellular phenotype by enabling the expression of a functional RAG1 protein. This makes the engineered RAG1-edited HSCs, respectively the population of cells of the present invention, particularly useful in the treatment of SCID related to RAG1. These RAG1-edited HSCs or population of cells according to the present invention may also be used in the manufacture of a medicament, such as a medicament for use in treatment of SCID related to RAG1.

The present invention thus provides an engineered RAG-1 edited HSC according to the invention for use in the treatment of SCID related to RAG1.

The present invention further provides an engineered RAG-1 edited HSC according to the invention for use in haematopoietic stem cell transplantation.

The present invention further provides a population of cells according to the invention for use in the treatment of SCID related to RAG1.

The present invention further provides a population of cells according to the invention for use in haematopoietic stem cell transplantation.

The present invention further provides a pharmaceutical composition comprising an engineered RAG1-edited HSC according to the invention, or a population of cells according to the invention, and a pharmaceutically acceptable excipient and/or carrier.

Suitably, such composition comprises an engineered RAG1-edited HSC of the invention, or the population of cells of the invention, in a therapeutically effective amount. An “effective amount” or “therapeutically effective amount” refers to that amount of a composition described herein which, when administered to a subject, is sufficient to provide a therapeutic or prophylactic benefit, i.e. aids in treating the disease. The amount of a composition that constitutes a “therapeutically effective amount” may vary depending on the cell preparations, the condition and its severity, the manner of administration, and the age of the subject to be treated, but can be determined routinely by one of ordinary skill in the art having regard to his own knowledge and to this disclosure.

The invention is thus more particularly drawn to a therapeutically effective population of engineered RAG-1 edited HSCs, wherein at least 20%, preferably 50%, more preferably 80% of the cells in said population have been modified according to any one of the methods described herein. Said therapeutically effective population of engineered HSCs, as per the present invention, comprises cells with a corrected endogenous RAG1 locus, allowing the expression of functional RAG1 protein.

Suitable pharmaceutically acceptable excipients and carriers are well known to the skilled person, and have been described in the literature, such as in Remington's Pharmaceutical Sciences, the Handbook of Pharmaceutical Additives or the Handbook of Pharmaceutical Excipients.

In some embodiments, the invention provides a cryopreserved pharmaceutical composition comprising: (a) a viable composition of engineered RAG1-edited HSCs of the present invention; (b) an amount of cryopreservative sufficient for the cryopreservation of the HSCs; and (c) a pharmaceutically acceptable carrier.

As used herein, “cryopreservation” refers to the preservation of cells by cooling to low sub-zero temperatures, such as (typically) 77 K or −196° C. (the boiling point of liquid nitrogen). At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature. The injurious effects associated with freezing can be circumvented by (a) use of a cryoprotective agent, (b) control of the freezing rate, and (c) storage at a temperature sufficiently low to minimize degradative reactions. Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-Sorbitol, D-mannitol, D-sorbitol, i-inositol, D-lactose, choline chloride, amino acids, methanol, acetamide, glycerol monoacetate, and inorganic salts. In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4° C. until freezing, since DMSO concentrations of about 1% are toxic at temperatures above 4° C.

The present invention provides the pharmaceutical composition of the present invention for use in the treatment of SCID related to RAG1.

The present invention also provides the pharmaceutical composition of the present invention for use in in hematopoietic stem cell transplantation.

The present invention also provides an isolated nucleic acid or vector of the invention, comprising the exogenous sequence as defined herein, for use in the treatment of SCID related to RAG1.

The present invention also provides the sequence specific reagent inducing DNA cleavage of the invention, the isolated nucleic acid of the invention encoding same or the vector encoding same for use in the treatment of SCID related to RAG1.

The present invention also provides the sequence specific reagent inducing DNA cleavage of the invention, the isolated nucleic acid of the invention encoding same or the vector encoding same in combination with an isolated nucleic acid or vector of the invention, comprising the exogenous sequence as defined herein, for use in the treatment of SCID related to RAG1.

The present invention also provides the kit of the invention for use in the treatment of SCID related to RAG1.

The present invention also provides the sequence specific reagent inducing DNA cleavage of the invention, the polynucleotide of the invention encoding same or the vector encoding same in combination with an isolated nucleic acid or vector of the invention, comprising the exogenous sequence as defined herein, for use in gene editing in vivo in HSCs, notably HSCs of a patient suffering from SCID related to RAG1.

The present invention further provides a method for treating a Severe Combined Immunodeficiency (SCID) related to RAG1 in a patient, such as a human patient, in need thereof, the method comprising administering an engineered RAG1-edited HSC of the invention, a population of cells of the invention or a pharmaceutical composition of the invention to said patient.

The present invention further provides a method for haematopoietic stem cell transplantation in a patient, such as a human patient, in need thereof, the method comprising administering an engineered RAG1-edited HSC of the invention, a population of cells of the invention or a pharmaceutical composition of the invention to said patient.

Generally, the treatment of (SCID) related to RAG1 according to the invention can be ameliorating, curative or prophylactic.

The administration of the engineered HSCs or population of cells according to the present invention may be carried out in any convenient manner, including injection, transfusion, implantation or transplantation. The engineered HSCs or population of cells according to the present invention may be administered to a patient by intravenous injection.

The administration of the HSCs or population of cells can consist of the administration of 10⁴-10⁸ gene edited cells per kg body weight, preferably 10′ to 10⁶ cells/kg body weight including all integer values of cell numbers within those ranges. The present invention thus can provide more than 10 doses comprising between 10⁴ to 10⁶ gene edited cells per kg body weight originating from a single patient's sampling.

The engineered HSCs or population of cells of the invention can be administrated in one or more doses. According to some embodiments, the therapeutic effective amount of cells is administrated as a single dose. According to some embodiments, the therapeutic effective amount of cells is administrated as more than one dose over a period time. Timing of administration is within the judgment of managing physician and depends on the clinical condition of the patient.

The dosage administrated will be dependent upon the age, health and weight of the patient receiving the treatment, the kind of concurrent treatment, if any, frequency of treatment and the nature of the effect desired.

### Certain Definitions

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.
As used herein, the term “locus” is the specific physical location of a DNA sequence (e.g. of a gene, such as the RAG1 gene) in a genome. The term “locus” can refer to the specific physical location of a rare-cutting endonuclease target sequence on a chromosome. Such a locus can comprise a target sequence that is recognized and/or cleaved by a sequence-specific reagent according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.
By “DNA target”, “DNA target sequence”, “target DNA sequence”, “nucleic acid target sequence”, or “target sequence” it is intended a polynucleotide sequence that can be targeted and processed by a sequence-specific reagent according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. The target sequence is defined by the 5′ to 3′ sequence of one strand of said target. Generally, the target sequence is adjacent or in the proximity of the locus to be processed either upstream (5′ location) or downstream (3′ location). In a preferred embodiment, the target sequences and the proteins are designed in order to have said locus to be processed located between two such target sequences. Depending on the catalytic domains of the proteins, the target sequences may be distant from 5 to 50 bases (bp), preferably from 10 to 40 bp, more preferably from 15 to 30, even more preferably from 15 to 25 bp. These later distances define the spacer referred to in the description and the examples. It can also define the distance between the target sequence and the nucleic acid sequence being processed by the catalytic domain on the same molecule.
As used herein, “exogenous” sequence generally refers to any nucleotide or nucleic acid sequence that was not initially present at the selected locus. By opposition “endogenous sequence” means a cell genomic sequence initially present at a locus. An “exogenous” sequence is thus a foreign sequence introduced into the cell, and thus allows distinguishing engineered cells over sister cells that have not integrated this exogenous sequence at the locus.
By “sequence-specific reagent inducing DNA cleavage” it is meant any active molecule that has the ability to specifically recognize a selected polynucleotide sequence in a genomic locus, preferably of at least 9 bp, more preferably of at least 10 bp and even more preferably of at least 12 bp in length, and that catalyzes the breakage of the covalent backbone of a polynucleotide. Non-limiting examples of a “sequence-specific reagent inducing DNA cleavage” according to the invention include reagents that have nickase or endonuclease activity. The sequence-specific reagent can be a chimeric polypeptide comprising a DNA binding domain and another domain displaying catalytic activity. Such catalytic activity can be for instance a nuclease to perform gene inactivation, or nickase or double nickase to preferentially perform gene insertion by creating cohesive ends to facilitate gene integration by homologous recombination, or to perform base editing as described in Komor et al. (2016) Nature 19; 533(7603):420-4.
The term “endonuclease” generally refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Precision genome surgery. Nat. Biotechnol. 25(7): 743-4).
The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
By “vector” is meant a nucleic acid molecule capable of transporting another nucleic acid to which it has been linked. A “vector” in the present invention includes, but is not limited to, a viral vector, a plasmid, a PCR product, a RNA vector or a linear or circular DNA or RNA molecule which may consist of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e. g. adeno-associated viruses, AAV), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomega-lovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “hematopoietic stem cells” (“HSCs”), it is meant multipotent stem cells derived from the bone marrow that have the capacity to self-renew and the unique ability to differentiate into all of the different cell types and tissues of the myeloid or lymphoid cell lineages, including but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSC also refers to long term repopulating HSC (LT-HSC) and short term repopulating HSC (ST-HSC). LT-HSC and ST-HSC are distinguished based on functional potential and on cell surface marker expression. For example, in some embodiments, human LT-HSCs are CD34+, CD38−, CD45RA−, CD90+, CD133+ and ST-HSCs are CD34+, CD38−, CD45RA−, CD90−, CD133−. In addition, ST-HSCs are less quiescent (i.e., more active) and more proliferative than LT-HSCs under homeostatic conditions. However, LT-HSCs have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in any of the methods described herein. In some embodiments, ST-HSC are useful because they are highly proliferative and thus, can more quickly give rise to differentiated progeny.
By “long term repopulating HSC” or “LT-HSC” it is meant a type of hematopoietic stem cells capable of maintaining self-renewal and multilineage differentiation potential throughout life. Phenotype markers characteristic for LT-HSCs include, but are not limited to, CD34+, CD38−, CD45RA−, CD90+, and CD133+.
By “primary cell” or “primary cells” it is meant cells taken directly from living tissue (e.g. biopsy material or blood sample) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.
By “originating from” it is meant that a cell or cells, such as HSCs, have been obtained from a patient suffering from SCID related to RAG1. In general, cells are provided from patients through a variety of methods known in the art, as for instance by leukapheresis techniques as reviewed by Schwartz J. et al. [Guidelines on the use of therapeutic apheresis in clinical practice-evidence-based approach from the Writing Committee of the American Society for Apheresis: the sixth special issue (2013) J. Clin. Apher. 28(3):145-284]. HSCs can be taken from bone marrow, and more particularly from the pelvis, at the iliac crest, using a needle or syringe. Alternatively, HSCs may be harvested from the circulating peripheral blood, while blood donors are injected with a HSC mobilizing agent, such as granulocyte-colony stimulating factor (G-CSF) and/or plerixafor, or CXCL2, that induces cells to leave the bone marrow and circulate in the blood vessels. HSCs may also be harvested from cord blood. HSCs may also be obtained from induced pluripotent stem (iPS) cells derived from the patient.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 80%, at least 85%, at least 90%, at least 95%, at least 98% or at least 99% identity to specific polypeptides described herein and preferably exhibiting substantially the same functions, as well as polynucleotide encoding such polypeptides, are contemplated. Unless otherwise stated, the present invention encompasses polypeptides and polynucleotides that have the same function and share at least 80%, generally at least 85%, preferably at least 90%, more preferably at least 95% and even more preferably at least 97% with those described herein.
The term “subject” or “patient” as used herein means a human, and more specifically a human suffering from SCID related to RAG1.
As used herein, the term “about” means plus or minus 10% of the numerical value of the number with which it is being used.

Where a numerical limit or range is stated herein, the endpoints are included. Also, all values and subranges within a numerical limit or range are specifically included as if explicitly written out.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps or components but do not preclude the addition of one or more additional features, steps, components or groups thereof.

The above written description of the invention provides a manner and process of making and using it such that any person skilled in this art is enabled to make and use the same, this enablement being provided in particular for the subject matter of the appended claims, which make up a part of the original description.

Having generally described this invention, a further understanding can be obtained by reference to certain specific examples, which are provided herein for purposes of illustration only, and are not intended to limit the scope of the claimed invention.

### EXAMPLES

### Example 1: Materials and Methods

### Cell Culture

### HSC

CD34⁺ hematopoietic stem cells (HSCs) from mobilized peripheral blood were purified using the CD34 MicroBead Kit UltraPure (Miltenyi Biotec, Cat. No. 130-100-453) according to the manufacturer's instruction. Purified CD34⁺ cells were frozen in CryoStor CS10 (StemCell Technologies, Cat. No. 07930) at a concentration of 1×10⁶ cells/ml and stored in liquid nitrogen until usage.

CD34⁺ cells were cultured in CellGenix GMP SCGM (CellGenix, Cat. No. 20806-0500) supplemented with recombinant human cytokines: SCF 300 ng/ml, Flt3-L 300 ng/ml, TPO 100 ng/ml, IL-3 60 ng/ml (SCF; Cat. No. 11343327, Flt3-L; Cat. No. 11343307, IL-3; Cat. No. 11340037, all Immunotools; TPO; Cat. No. 300-18, PeproTech) for three days prior to electroporation and transduction. For flow cytometry analysis experiments, medium was further supplemented with small molecules: StemRegenin 1 (StemCell Technologies; Cat. No. 72342) at 750 nM, and UM171 (StemCell Technologies; Cat. No. 72912) at 35 nM. After electroporation and transduction, IL-3 was removed from the culture medium. The cells were cultured for additional 48h after treatment and harvested. Cells were cultured at 37° C. with 5% CO₂ if not otherwise indicated.

### Jurkat

Jurkat cells (ATCC; Clone E6-1) were cultured in RPMI medium (Gibco, Cat. No. 11879020) supplemented with 10% FCS (Cytiva, Cat. No. SH30070.02) and 1% of penicillin/streptomycin (Sigma-Aldrich, Cat. No. P4333).

### Primary T Cells

Cryopreserved human PBMCs were acquired from ALLCELLS (cat #PB006F). PBMCs were cultured in X-vivo-15 media (Lonza, #BE04-418Q), containing IL-2 (Miltenyi, #130-097-748), and human serum AB (Gemini, #100-318). Human T activator CD3/CD28 dynabeads (Thermo Fisher Scientific) or TransAct (Miltenyi, #130-097-743) was used to activate T-cells.

Gene Editing Reagents and mRNA Productions:

RAG1-specific TALE-nucleases, TALEN-2 (SEQ ID NO: 2 and SEQ ID NO: 3), TALEN-6 (SEQ ID NO: 4 and SEQ ID NO: 5), TALEN-A (SEQ ID NO: 6 and SEQ ID NO: 7), TALEN-B (SEQ ID NO: 8 and SEQ ID NO: 9), GSE56 (SEQ ID NO: 10) and 53BP1 inhibitor (SEQ ID NO: 11) encoding mRNAs were produced using the HiScribe T7 ARCA mRNA Kit (NEB, Cat. No. E2065S) following the manufacturer's instructions.

The nucleic acid sequences encoding the RAG1-specific TALE-nucleases were as follows: SEQ ID NO: 30 and SEQ ID NO: 31 for TALEN-2, SEQ ID NO: 32 and SEQ ID NO: 33 for TALEN-6, SEQ ID NO: 34 and SEQ ID NO: 35 for TALEN-A, and SEQ ID NO: 36 and SEQ ID NO: 37 for TALEN-B. The monomers of TALEN-2 target a nucleic acid sequence of SEQ ID NO: 39 and SEQ ID NO: 40, respectively, and the monomers of TALEN-6 target a nucleic acid sequence of SEQ ID NO: 42 and SEQ ID NO: 43, respectively. The monomers of TALEN-A target a nucleic acid sequence of SEQ ID NO: 44 and SEQ ID NO: 45, respectively, and the monomers of TALEN-B target a nucleic acid sequence of SEQ ID NO: 46 and SEQ ID NO: 47, respectively.

The nucleic acid sequences encoding GSE56 was SEQ ID NO: 48 and the nucleic acid sequence encoding 53BP1 inhibitor was SEQ ID NO: 49.

### Exogenous Polynucleotide Donor Template Constructs:

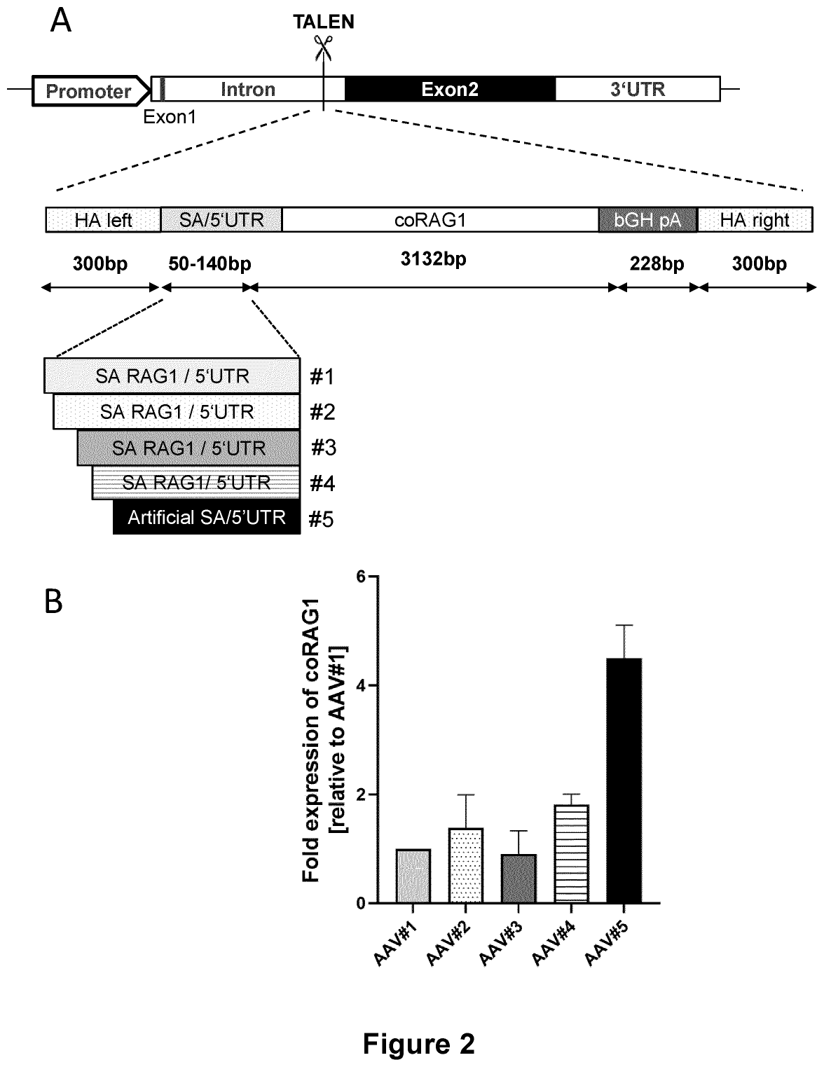

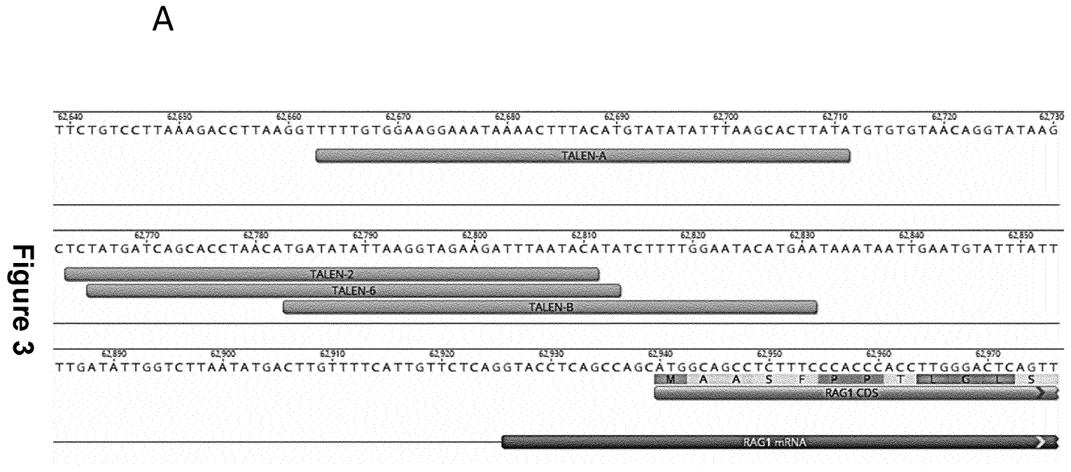

Various donor templates comprising the codon-optimized RAG1 sequence of SEQ ID NO: 19 to be inserted at RAG1 loci were created and vectorized in AAV6 particles by Vigene. These templates are differing only in the region upstream of the codon-optimized RAG1 sequence. Constructs #1 to #4 contain parts of the endogenous RAG1 intron sequence preceding exon 2 in varying lengths (50-140 bp). Construct #5 contains a short sequence harbouring a synthetic splice acceptor site (SEQ ID NO: 12) (FIG. 2A).

### Gene Editing Protocols

### HSCs

For gene-editing, 1×10⁶ cells were harvested (300×g, 10 min) and the medium removed. CD34⁺ cells were resuspended in 50 μl of CliniMACS Electroporation Buffer (Miltenyi Biotec, Cat. No. 170-076-625). The cells were subsequently mixed briefly with pre-mixed mRNAs (1 μg of each TALEN mRNA with/without 2 μg of GSE56 mRNA and/or 4 μg of 53BP1 inhibitor mRNA), transferred to a 2 mm electroporation cuvette (VWR, Cat. No. 732-1136) and electroporated using a CliniMACS Prodigy device (Miltenyi Biotec). Cells were immediately resuspended in 0.5 ml of complete medium (without IL-3) and split into wells of 96-well plate (0.25×10⁶ cells/well). The cells were recovered for 15 min at 37° C. prior to addition of AAV6 particles at a concentration of 1×10¹ genome copies (GC)/cell. Samples were cultured at 32° C. for 24h. Then, 100 μl of fresh medium was added and cells cultured for 24h at 37° C. prior to harvest (300×g, 10 min) and genomic DNA extraction.

### Jurkat

0.8×10⁶ cells were harvested (300×g, 5 min) and medium removed. Transfer of TALEN mRNAs was performed via nucleofection using a Lonza 4D-Nucleofector device in combination with the SE Cell Line 4D-Nucleofector X Kit S (Lonza, Cat. No. V4XP-1032) following the manufacturer's instructions. Briefly, cells were resuspended in 20 μl of supplemented SE Cell Line Solution, combined with pre-mixed TALEN mRNAs (1 μg of each TALEN mRNA), transferred to an electroporation cuvette and electroporated using program CK116. The nucleofected cells were incubated for 15 min at room temperature. The cuvette was washed with 100 μl of pre-warmed medium and cells split into eight wells of a 96-well culture plate with 1×10⁵ cells/well in 150 μl of medium. Cells were let to recover for 15 min at 37° C. prior to addition of AAV6 particles at a concentration of 1×10⁵ GC/cell. Cells were cultured at 32° C. for 24h before being incubated at 37° C. for an additional 72h prior to harvest (300×g, 5 min) and gDNA extraction.

Primary T cells For high throughput screening, 4 days post activation 1×10⁶ PBMCs were electroporated with 2 μg (1 μg per TALEN arm) mRNA using a 4D-Nucleofector/96-well Shuttle Device (Lonza, #AAM-1001S and #AAF-1002B), according to the manufacturer's protocol. Transfected cells were then incubated overnight at 30° C., 5% CO₂ in complete medium (X-Vivo 15 media supplemented with 20 ng/mL human IL-2 and 5% human AB serum). Transfected cells were then cultured at 37° C., 5% CO₂ in complete medium for an additional 48h.

For TALEN validation, four days post activation 5×10⁶ PBMCs were electroporated with 5-10 μg (2.5-5 μg per TALEN arm) mRNA using an AgilePulse MAX system (Harvard Apparatus). Transfected cells were incubated overnight at 30° C., 5% CO₂ in complete medium (X-Vivo 15 media supplemented with 20 ng/mL human IL-2 and 5% human AB serum). One day post transfection, cells were split into fresh complete media at a density of 1×10⁶ cell/ml and cultured for 48h at 37° C., 5% CO₂.

Three days post transfection, cells were then collected and gDNA purified as per the manufacturer's instructions (Qiagen, #69504).

### TALEN Activity Evaluations.

For the T7E1 assay, targeted PCRs were performed (NEB, M0271 L) with custom oligonucleotides (Integrated DNA Technologies SEQ ID NO: 50 and SEQ ID NO: 51 for TALEN-2). PCR products were then unannealed/reannealed in annealing buffer (annealing buffer 10×: Tris 10 mM, EDTA 1 mM, NaCl 0.1 M) with the following steps: 1) 95° C.-10 min, 2) Down to 85° C. at 3° C./s, and 3) Down to 25° C. at 0.3° C./s. The reannealed product was then digested using the T7E1 enzyme in NE2 buffer for 15 min at 37° C. and analysed on 5% precast polyacrylamide gels. To assess TALEN-mediated indel formation at the targeted locus, targeted PCRs were first performed with primers (SEQ ID NO: 52 and SEQ ID NO: 53 for TALEN-6, SEQ ID NO: 54 and SEQ ID NO: 55 for TALEN-A and SEQ ID NO: 56 and SEQ ID NO: 57 for TALEN-B) followed by a second indexing PCR (NEB, M0531 L), both using custom oligonucleotides (Integrated DNA Technologies). NGS sequencing was carried out using the 2×250 cycles Illumina MiSeq v2 Reagent Kit (Illumina, #15033625) according to Illumina's MiSeq System User Guide. The sequences of the primers used are provided in Table 1.

TABLE 1
Pairs of primers used to assess TALEN activity

| | Sequences | |
|---|---|---|
| | (5′-3′) of | SEQ |
| Targeted | left and right | ID |
| locus | oligonucleotides | NO: |
| TALEN-2 | GCAAAGATGAATCAAAGATTCTG | 50 |
| | CTTCAGGTGTCTTTTCAAAGG | 51 |
| TALEN-6 | AGCACCTAATGTATACTGGGACC | 52 |
| | GCTGGCTGAGGTACCTGAGA | 53 |
| TALEN-A | AGCACCTAATGTATACTGGGACC | 54 |
| | GCTGGCTGAGGTACCTGAGA | 55 |
| TALEN-B | AGCACCTAATGTATACTGGGACC | 56 |
| | GCTGGCTGAGGTACCTGAGA | 57 |

Quantification of coRAG1 Expression in Jurkat Cells by RT-qPCR.

Gene edited Jurkat cells were subjected to RNA extraction using the RNeasy Mini Kit (Qiagen, Cat. No. 74104) following the manufacturer's instructions. From the extracted RNA, cDNA was produced by reverse transcription using the QuantiTect Reverse Transcription Kit (Qiagen, Cat. No. 205314) according to the provided manual. Expression of coRAG1 was quantified by RT-qPCR. To this end, samples were analysed on a MasterCycler RealPlex 4 Gradient S device (Eppendorf) using the Luna® Universal qPCR Master Mix (NEB, Cat. No. M3003E) according to the manufacturer's instructions. Used primers bind to exon 1 (#5278) and coRAG1 (#4938).

### Colony Forming Unit (CFU) Assay.

CFU assay was performed according to the instructions provided by StemCell Technologies. Onto a single 3.5 cm CFU assay dish, 500-2500 cells were seeded in MethoCult™ H4034 Optimum matrix (StemCell Technologies, Cat. No. 04044) after dilution in IMDM (StemCell Technologies, Cat. No. 07700) using a 3 cc Syringe (StemCell Technologies, Cat. No. 28230) and Blunt-End Needles (StemCell Technologies, Cat. No. 28110). Cells were cultured at 37° C. with 5% CO₂ for 14-17 days prior to analysis of colony numbers and lineage distribution, as well as harvesting of single colonies for further analysis.

### Genotyping of CFU Colonies.

Single colonies were picked from the CFU assay plates, transferred into PCR tubes and resuspended in 20 μl of Lysis buffer containing 19.6 μl of DirectPCR Lysis Reagent (Viagen, Cat. No. 301-C) and 0.4 μl of Proteinase K (Thermo Scientific, Cat. No. E00491). Lysis was performed in a TProfessional TRIO Thermocycler (Biometra) by incubation at 56° C. for 90 min followed by inactivation of the Lysis buffer at 85° C. for 45 min. Of the cell lysate, 9 μl were used as a template for a single PCR reaction. In each reaction, three primers were used to discriminate between integration/non-integration of coRAG1 at the 5′ end (with #4938) and 3′ end (with #4939) as described in Table 2.

TABLE 2
PCR screening for integration/non integration detection

| | Volume |
|---|---|
| | [μl] |
| Lysate | 9 |
| 5X Q5 Reaction Buffer | 10 |
| Q5 Hot Start High-Fidelity | 0.5 |
| DNA Polymerase | |
| 40 mM dNTPs | 1 |
| 10 μM #4937 | 1 |
| 10 μM #4938 or #4939 | 1 |
| 10 μM #4940 | 1 |
| H₂O | up to 50 μl |

Quantification of Knock-In Efficiency by ddPCR.

The ddPCR assay to measure knock-in efficiency in HSCs was performed basically according to the manufacturer's instructions. Briefly, 20-50 ng of gDNA were combined in a PCR reaction containing QX200 ddPCR EvaGreen Supermix (Bio-Rad, Cat. No. #1864034) and either of the three primer pairs #4937/#4938 (5′ junction), #4939/4940 (3′ junction), or #4948/#5020 (reference) as shown on Table 3.

TABLE 3
ddPCR condition assay

| | Volume |
|---|---|
| | [μl] |
| gDNA (20-50 ng) | X |
| 2X ddPCR EvaGreen Supermix | 10 |
| 2 μM fwd primer | 1 |
| 2 μM rev primer | 1 |
| H₂O | up to 20 μl |

Droplets were generated using QX200 Droplet Generation Oil for EvaGreen (Bio-Rad, Cat. No. #1864006) in DG8 Cartridges (Bio-Rad, Cat. No. #1864008) covered by DG8 Gaskets (Bio-Rad, Cat. No. #1864009) using a QX200 Droplet generator (Bio-Rad). Droplets were transferred into ddPCR 96-Well Plates (Bio-Rad, Cat. No. #12001925), plates sealed using a PX1 PCR plate sealer (Bio-Rad) and piercable foil heat seals (Bio-Rad, Cat. No. #1814040). The PCR reaction was performed in a standard PCR cycler using the program “ddPCR” provided in Table 4:

TABLE 4
ddPCR conditions

| | Temp. | Time | | | |
|---|---|---|---|---|---|
| Step | [° C.] | [min] | go to | Loops | ΔT/s |
| 1 | 95 | 05:00 | | | 1 |
| 2 | 95 | 00:30 | | | 1 |
| 3 | 62 | 01:00 | | | 1 |
| 4 | 72 | 02:00 | Step 2 | 49 | 1 |
| 5 | 4 | 05:00 | | | 1 |
| 7 | 90 | 05:00 | | | 1 |
| 8 | 12 | hold | | | 1 |

Droplets were read on a QX200 Droplet reader (Bio-Rad) and the data analyzed using QuantaSoft Analysis Pro 1.0.596.

### Analysis of HSC Subpopulations.

HSC subpopulations were sorted by FACS using a MoFlo Astrios (Beckman Coulter). 6×10⁶ cells were stained with the antibody mix listed in Table 5 for 60-90 min on ice, resuspended in 200-300 μl FACS buffer consisting of PBS supplemented with 5% FCS (Cytiva, Cat. No. SH30070.02).

TABLE 5
Antibodies used in this study

| | | Company/Cat. | | Volume |
|---|---|---|---|---|
| Epitope | Clone | No. | Dye | [μl] |
| human CD34 | 581 | BioLegend/343514 | APC/Cyanine7 | 10-20 |
| human CD90 | 5E10 | BD Pharmingen/559869 | APC | 10-20 |
| human CD45RA | HI100 | BioLegend/304106 | FITC | 10-20 |
| human CD133/1 | REA753 | Miltenyi/130-111-079 | PE | 5-10 |
| human CD38 | HB-7 | BioLegend/356608 | PE/Cyanine7 | 10-20 |

After staining, cells were washed three times with 1 ml of FACS buffer (300×g, 10 m), resuspended in 300-500 μl of FACS buffer and sorted. Long-term repopulating hematopoietic stem cells (LT-HSCs) were discriminated by the phenotype CD34+/CD38−/CD45RA−/CD90+/CD133-1+, short-term repopulating hematopoietic stem cells (ST-HSCs) by the phenotype CD34+/CD38−/CD45RA−/CD90−/CD133-1−.

TABLE 6
Oligonucleotides used in this study.

| | | SEQ |
|---|---|---|
| | | ID |
| Number | Sequence (5′-3′) | NO: |
| #5278 | TGCAGACATCTCAACACTTTGGC | 58 |
| #4937 | AACCAACCCCCTGGAAGACTG | 59 |
| #4938 | GGATGAAAGACCCAGCGTAGG | 60 |
| #4939 | TGGGAAGACAATAGCAGGCATG | 61 |
| #4940 | GAGATTGCTCCAGAGAGGGTTTCC | 62 |
| #4948 | AACTTCAGTAAAGCGGGGACTTG | 63 |
| #5020 | TGAGTTGTAATGCACAGATCGCTAG | 64 |

### Example 2: Screening of TALEN Targeting RAG1 Intron 1

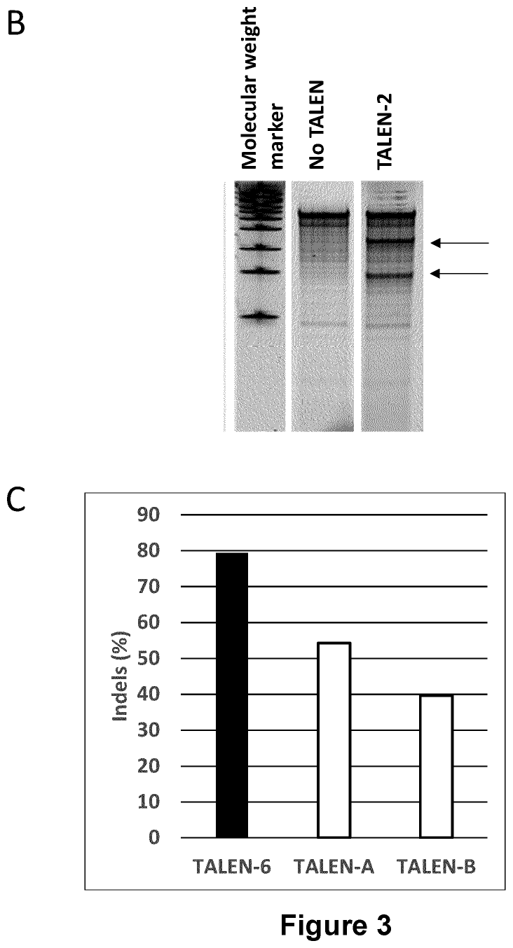

Several TALEN targeting intron1 of RAG1 (4 of them presented in FIG. 3A) were designed, produced in a high throughput format and screened in primary T cells for their cleavage activity measured with either a T7E1 or for a more precise validation by Next Generation Sequencing assay. FIG. 3B shows one of the best TALEN (TALEN-2) identified with this screening. FIG. 3C reveals that TALEN-6, another very good TALEN identified, was able to induce around 80% of indels (insertion/deletion) events while TALEN-A and TALEN-B were able to induce around 50% or 40% of indels.

### Example 3: Targeted Integration of coRAG1 Allowed RAG1 Expression in Jurkat Cell Line

To trigger integration of coRAG1, two TALEN pairs (TALEN-2 and TALEN-6) were first tested in combination with the transduced coRAG1 via electroporation of mRNA into Jurkat, followed by transduction with AAV6 vectors comprising coRAG1 donor templates. To ensure correct splicing and efficient transcription of the corrective DNA, various donor templates differing only in the region upstream of the codon-optimized RAG1 sequence, were tested. Constructs #1-#4 contain parts of the endogenous RAG1 intron 1 sequence preceding exon2 in varying lengths (50-140 bp). Construct #5 contains a short sequence harbouring a synthetic splice acceptor site (FIG. 2A).

Expression of all five constructs was tested in Jurkat cells as surrogate. These cells, in contrast to primary HSCs, constitutively express the RAG1 locus. Surprisingly, while all constructs containing the endogenous splice acceptor site (#1-#4) showed similar, moderate expression of coRAG1, expression was enhanced by more than 4-fold in cells transduced with AAV6 donor #5 (“AAV #5”) relative to construct #1 (“AAV #1”) (FIG. 2B).

### Example 4: Targeted Integration of coRAG1 in HSCs

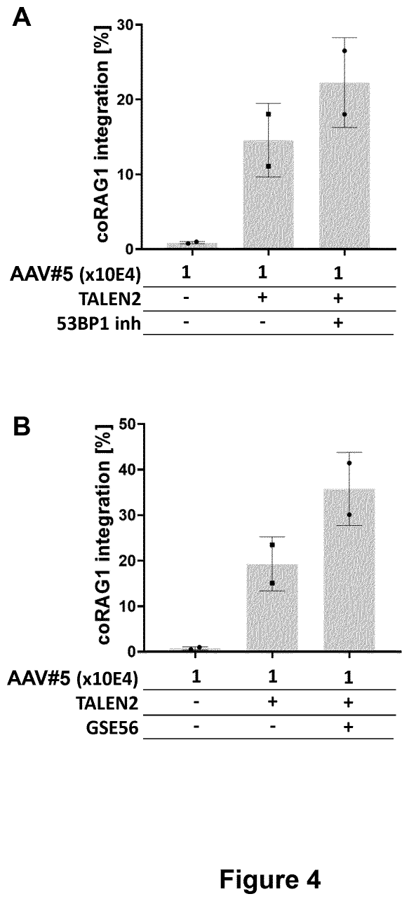

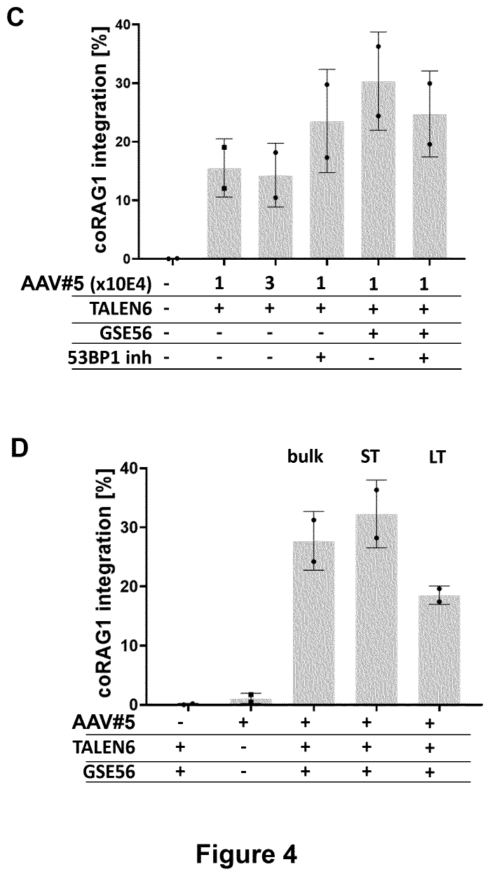

Having identified AAV #5 as superior donor template, integration of this construct was tested in primary human HSCs in combination with TALEN-2 and TALEN-6. Transduction of cells with 1×10⁴ GC/cell after TALEN-mediated cleavage, yielded integration of coRAG1 in 15-20% of RAG1 alleles for both TALENs (FIGS. 4A and 4C). Increasing the number of AAV6 particles did not improve integration efficacy (FIG. 4C).

To leverage integration, inhibitors 53BP1 and/or GSE56 were expressed. While 53BP1 inhibitor inhibits 53BP1, a factor involved in non-homologous end-joining (NHEJ) based DNA-repair (Canny et al., 2018, Nat. Biotechnol. 36(1): 95-102), GSE56 inhibits the cell-cycle control protein p53 (Ossovskaya et al., 1996, Proc. Natl. Acad. Sci. U.S.A. 93: 10309-10314). Combining mRNA encoding 53BP1 inhibitor with TALEN mRNA improved coRAG1 integration by ˜1.5-fold, with integration in ˜22% of alleles when using TALEN-2 (FIG. 4A) and ˜23% with TALEN-6 (FIG. 4C), respectively. A ˜2-fold increase in targeted integration was observed when GSE56 was used, with integration frequencies of ˜30-35% of RAG1 alleles (FIGS. 4B and C). When both factors were combined, no additive effect was observed (FIG. 4C).

A hallmark for a life-long cure is the correction of self-renewing long-term repopulating hematopoietic stem cells (LT-HSCs). Integration of coRAG1 in this subpopulation was determined using optimized conditions that included GSE56. Sorting of edited cells by FACS into LT-HSCs and short-term HSCs (ST-HSCs) subpopulations with subsequent analysis of coRAG1 integration revealed slightly higher levels of targeted integration in ST-HSCs (32%) compared to bulk edited cells (28%). While the editing frequencies were lower in the naïve LT-HSCs population (19%) (FIG. 4D).

### Example 5: Clonal Analysis of Gene-Edited HSCs

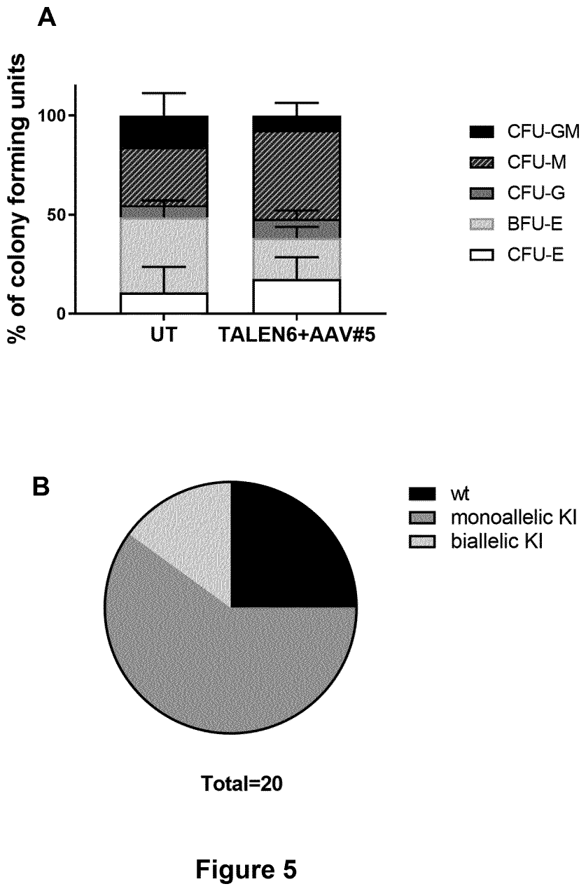

In order to test whether the full differentiation potential was preserved in gene-edited cells, a colony-forming unit (CFU) assay was performed on untreated or TALEN-6 and AAV #5 treated HSCs. Outgrowth of all colony types at a frequency highly similar to that observed in untreated cells confirmed that integration of coRAG1 in a large proportion of cells did not imbalance the overall differentiation profile (FIG. 5A).

To confirm stability of coRAG1 knock-in, individual colonies were collected from CFU assay plates and analyzed for the presence of integrated donor template by PCR that allowed discriminating between mono- and bi-allelic knock-in. Twenty individual clones were analyzed, ten of which had been treated with TALEN and AAV #5 donor, while the remaining ten clones were treated with TALEN plus 53BP1 inhibitor plus AAV #5 donor. The presence of integrated coRAG1 was confirmed in 15 (75%) of all clones. The majority of clones (60%) displayed mono-allelic editing while targeted integration in both alleles was achieved in 3 clones (15%) (FIG. 5B). This shows that gene edited cells did not have a general growth disadvantage and that the knock-in achieved might potentially be even higher than determined by ddPCR (FIG. 4A-D), thus increasing the potential clinical benefits.

### LIST OF CERTAIN REFERENCES CITED IN THE DESCRIPTION

[1] Ghosh, S., Thrasher, A. J. & Gaspar, H. B. Gene therapy for monogenic disorders of the bone marrow. Br J Haematol (2015).
[2] Corneo, B. et al. Identical mutations in RAG1 or RAG2 genes leading to defective V(D)J recombinase activity can cause either T-B-severe combined immune deficiency or Omenn syndrome. Blood 97, 2772-2776 (2001).
[3] Lagresle-Peyrou, C. et al. Long-term immune reconstitution in RAG-1-deficient mice treated by retroviral gene therapy: a balance between efficiency and toxicity. Blood 107, 63-72 (2006).
[4] Pike-Overzet, K. et al. Correction of murine Rag1 deficiency by self-inactivating lentiviral vector-mediated gene transfer. Leukemia 25, 1471-1483 (2011).
[5] Notarangelo, L. et al. Human RAG mutations: biochemistry and clinical implications. Nature Reviews Immunology 16, 234-246 (2016).
6] Owens, B. et al. Transcription activator like effector (TALE)-directed piggyBac transposition in human cells. Nucleic Acids Res. 41(19), 9197-207 (2013)
[7] Voigt, K. et al. Retargeting Sleeping Beauty Transposon Insertions by Engineered Zinc Finger DNA-binding Domains. Molecular Therapy 20(10), 1852-1862 (2012)
[8] Bhatt S. and Chalmers R. Targeted DNA transposition in vitro using a dCas9-transposase fusion protein. Nucleic Acids Res. 47(15), 8126-8135 (2019)
[9] Komor et al. Programmable editing of a target base in genomic DNA without double-stranded DNA cleavage. Nature 533(7603), 420-424 (2016)
[10] Urnov F., et al. Highly efficient endogenous human gene correction using designed zinc-finger nucleases. Nature 435, 646-651 (2005)
[11] Mussolino et al. A novel TALE nuclease scaffold enables high genome editing activity in combination with low toxicity. Nucl. Acids Res. 39(21), 9283-9293 (2011)
[12] Boissel et al. MegaTALs: a rare-cleaving nuclease architecture for therapeutic genome engineering. Nucleic Acids Research 42 (4), 2591-2601 (2013)
[13] Mussolino et al. TALEN® facilitate targeted genome editing in human cells with high specificity and low cytotoxicity. Nucl. Acids Res. 42(10), 6762-6773 (2014)
[14] Doudna, J. et al. The new frontier of genome engineering with CRISPR-Cas9. Science 346 (6213), 1077 (2014)
[15] Zetsche, B. et al. Cpf1 Is a Single RNA-Guided Endonuclease of a Class 2 CRISPR-Cas System. Cell 163(3), 759-771 (2015)
[16] Sather, B. D. et al. Efficient modification of CCR5 in primary human hematopoietic cells using a megaTAL nuclease and AAV donor template. Science translational medicine, 7(307), 307ra156 (2015)

1. An engineered RAG1-edited Haematopoietic Stem Cell (HSC) comprising an exogenous sequence comprising, from its 5′ to 3′ ends, the nucleic acid sequence SEQ ID NO: 12, a nucleic acid sequence encoding a functional RAG1 protein, and a polyA signal, wherein said exogenous sequence is integrated in said HSC's genome into Intron 1 of a non-functional endogenous RAG1 locus.

2. (canceled)

3. (canceled)

4. The engineered HSC according to claim 1, wherein said exogenous sequence comprises the nucleic acid sequence SEQ ID NO: 23, SEQ ID NO: 25, SEQ ID NO: 69, or SEQ ID NO: 72.

5. A population of cells comprising engineered HSCs according to claim 1.

6. A population of cells comprising engineered HSCs to claim 1, comprising at least 1%, at least 5%, at least 10%, at least 15%, at least 20%, at least 25%, at least 30%, at least 35%, or at least 40% of said engineered RAG1-edited HSCs.

7. (canceled)

8. (canceled)

9. A TALE-Nuclease heterodimer selected from:
(i) a TALE-Nuclease heterodimer targeting the polynucleotide sequence of SEQ ID NO: 41, comprising a first monomer targeting the polynucleotide sequence of SEQ ID NO: 42, and a second monomer targeting the polynucleotide sequence of SEQ ID NO: 43;
(ii) a TALE-Nuclease heterodimer targeting the polynucleotide sequence of SEQ ID NO: 41, comprising a first monomer comprising the amino acid sequence of SEQ ID NO: 4 or a variant thereof comprising an amino acid sequence having at least 80% sequence identity with SEQ ID NO: 4 and targeting the sequence of SEQ ID NO: 42, and a second monomer comprising the amino acid sequence of SEQ ID NO: 5 or a variant thereof comprising an amino acid sequence having at least 80% sequence identity with SEQ ID NO: 5 and targeting the sequence of SEQ ID NO: 43;
(iii) a TALE-Nuclease heterodimer targeting the polynucleotide sequence of SEQ ID NO: 41, comprising a first monomer comprising the amino acid sequence of SEQ ID NO: 4 and a second monomer comprising the amino acid sequence of SEQ ID NO: 5;
(iv) a TALE-Nuclease heterodimer targeting the polynucleotide sequence of SEQ ID NO: 38, comprising a first monomer targeting the polynucleotide sequence of SEQ ID NO: 39, and a second monomer targeting the polynucleotide sequence of SEQ ID NO: 40;
(v) a TALE-Nuclease heterodimer targeting the polynucleotide sequence of SEQ ID NO: 38, comprising a first monomer comprising the amino acid sequence of SEQ ID NO: 2 or a variant thereof comprising an amino acid sequence having at least 80% sequence identity with SEQ ID NO: 2 and targeting the sequence of SEQ ID NO: 39, and a second monomer comprising the amino acid sequence of SEQ ID NO: 3 or a variant thereof comprising an amino acid sequence having at least 80% sequence identity with SEQ ID NO: 3 and targeting the sequence of SEQ ID NO: 40; and
(vi) a TALE-Nuclease heterodimer targeting the polynucleotide sequence of SEQ ID NO: 38, comprising a first monomer comprising the amino acid sequence of SEQ ID NO: 2 and a second monomer comprising the amino acid sequence of SEQ ID NO: 3.

10. (canceled)

11. (canceled)

12. (canceled)

13. (canceled)

14. (canceled)

15. (canceled)

16. (canceled)

17. An isolated nucleic acid or vector encoding a TALE-Nuclease heterodimer according to claim 9.

18. (canceled)

19. An isolated nucleic acid or a vector comprising an exogenous sequence comprising, from its 5′ to 3′ ends, the nucleic acid sequence SEQ ID NO: 12, a nucleic acid sequence encoding a functional RAG1 protein, and a polyA signal, wherein said exogenous sequence is placed between a left homologous region at its 5′ end and a right homologous region at its 3′ end, homologous regions being relative to parts of intron 1 of SEQ ID NO: 28 of RAG1.

20. The population of cells according to claim 9, comprising an exogenous sequence comprising, from its 5′ to 3′ ends, the nucleic acid sequence SEQ ID NO: 12, a nucleic acid sequence encoding a functional RAG1 protein, and a polyA signal.

21. (canceled)

22. A pharmaceutical composition comprising an engineered HSC according to-claim 1, and a pharmaceutically acceptable excipient and/or carrier.

23. A method of treatment of a Severe Combined Immunodeficiency (SCID) related to RAG1 in a patient in need thereof comprising administering a therapeutically effective amount of an engineered RAG1-edited Haematopoietic Stem Cell (HSC) according to claim 1.

24. (canceled)

25. A kit comprising:
(i) at least one isolated nucleic acid or vector comprising an exogenous sequence comprising, from its 5′ to 3′ ends, the nucleic acid sequence SEQ ID NO: 12, a nucleic acid sequence encoding a functional RAG1 protein, and a polyA signal, wherein said exogenous sequence is placed between a left homologous region at its 5′ end and a right homologous region at its 3′ end, homologous regions being relative to parts of intron 1 of SEQ ID NO: 28 of RAG1, and
(ii) at least one isolated nucleic acid or vector encoding a TALE-nuclease heterodimer according to claim 9.

26. The kit according to claim 25 comprising an isolated nucleic acid or vector comprising the nucleic acid sequence SEQ ID NO: 18, SEQ ID NO: 26, SEQ ID NO: 70, or SEQ ID NO: 73, and an isolated nucleic acid or vector comprising a nucleic acid sequence encoding the amino acid sequences of SEQ ID NO: 4 and SEQ ID NO: 5.

27. (canceled)

28. A method of preparing engineered RAG1 edited haematopoietic stem cells comprising introducing into the HSCs to be engineered:
(i) at least one isolated nucleic acid or vector comprising an exogenous sequence comprising, from its 5′ to 3′ ends, the nucleic acid sequence SEQ ID NO: 12, a nucleic acid sequence encoding a functional RAG1 protein, and a polyA signal, wherein said exogenous sequence is placed between a left homologous region at its 5′ end and a right homologous region at its 3′ end, homologous regions being relative to parts of intron 1 of SEQ ID NO: 28 of RAG1; and
(ii) at least one isolated nucleic acid or vector coding for a sequence specific reagent inducing DNA cleavage that is capable of targeting and cleaving a sequence within intron 1 of SEQ ID NO: 28 of the endogenous RAG1 locus of said HSCs;
whereby engineered RAG1 edited haematopoietic stem cells are obtained.

29. The method according to claim 28, wherein said sequence specific reagent inducing DNA cleavage is the TALE-nuclease heterodimer according to claim 9.

30. (canceled)

31. The method according to claim 28, wherein said nucleic acid or vector of (i) comprises an exogenous sequence comprising the nucleic acid sequence SEQ ID NO: 18, SEQ ID NO: 26, SEQ ID NO: 70, or SEQ ID NO: 73, and wherein said nucleic acid or vector of (ii) comprises a nucleic acid sequence encoding the amino acid sequences of SEQ ID NO: 4 and SEQ ID NO: 5.

32. The method according to claim 28, wherein said HSCs are isolated from a tissue sample from a human patient suffering from Severe Combined Immunodeficiency (SCID) related to RAG1.

33. The method according to claim 28, wherein said HSCs are isolated from a tissue sample from a human patient suffering from Severe Combined Immunodeficiency (SCID) related to RAG1 and said tissue sample is a peripheral blood sample.

34. The TALE-Nuclease heterodimer according to claim 9, wherein said TALE-nuclease heterodimer targets: (a) the polynucleotide sequence of SEQ ID NO: 41, comprising a first monomer comprising the amino acid sequence of SEQ ID NO: 4 and a second monomer comprising the amino acid sequence of SEQ ID NO: 5; or (b) the polynucleotide sequence of SEQ ID NO: 38, comprising a first monomer comprising the amino acid sequence of SEQ ID NO: 2 and a second monomer comprising the amino acid sequence of SEQ ID NO: 3.


SEQUENCE LISTING (73 sequences)

SEQ ID NO: 1 - protein, 1043 aa, homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 2 - protein, 925 aa, artificial
  TALEN-2-Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 3 - protein, 925 aa, artificial
  TALEN-2-Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 4 - protein, 925 aa, artificial
  TALEN-6-Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 5 - protein, 925 aa, artificial
  TALEN-6-Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 6 - protein, 925 aa, artificial
  TALEN-A-Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 7 - protein, 925 aa, artificial
  TALEN-A-Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 8 - protein, 925 aa, artificial
  TALEN-B-Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 9 - protein, 925 aa, artificial
  TALEN-B-Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 10 - protein, 96 aa, artificial
  GSE56
MGCPGRDRRTEEENLRKKGEPHHELPPGSTKRALPNNTSSSPQPKKKPLDGEYFTLQIRGRERFEMFRELNEALELKDAQAGKEPGGSRAHSSHLK

SEQ ID NO: 11 - protein, 74 aa, artificial
  53BP1 inhibitor
MLIFVKTLTGKTITLEVEPSDTIENVKAKIQDKEGIPPDQQRLAFAGKSLEDGRTLSDYNILKDSKLHPLLRLR

SEQ ID NO: 12 - DNA, 54 bp, artificial
  Artificial Splice Acceptor
acaagctaggcccttttgctaatcatgttcatactgtcccttttttttccacag

SEQ ID NO: 13 - DNA, 3132 bp, homo sapiens
atggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaa

SEQ ID NO: 14 - DNA, 4103 bp, artificial
  AAV#1
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagtatcttttggaatacatgaataaataattgaatgtatttatttttattatttataagatacatcagtgggatattgatattggtcttaatatgacttgttttcattgttctcaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 15 - DNA, 4075 bp, artificial
  AAV#2
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacatgaataaataattgaatgtatttatttttattatttataagatacatcagtgggatattgatattggtcttaatatgacttgttttcattgttctcaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 16 - DNA, 4035 bp, artificial
  AAV#3
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgataaagatacatcagtgggatattgatattggtcttaatatgacttgttttcattgttctcaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 17 - DNA, 4005 bp, artificial
  AAV#4
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacttaatatgacttgttttcattgttctcaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 18 - DNA, 4028 bp, artificial
  donor template AAV#5
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 19 - DNA, 3146 bp, artificial
  coRAG1
gtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaa

SEQ ID NO: 20 - DNA, 3146 bp, artificial
  coRAG1bis
gtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagcaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccgatacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagcaaggagatcttcgtgcacataaataaagggggccgaccgcggcagcatttgttgagtcttacccgccgagcacaaaagcaccgacttcgagaactgaaactccaagttaaggcgttcgcagataaggaggagggaggggacgtaaaatccgtttgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttcgaaacgccgagaaagtgttgctgcctgggtatcatcactttgaatggcagcctccgctgaaaaacgtttcttcaagcaccgacgttggtatcatagacggcctgtctggcctgtcaagcagtgtggatgattaccctgtagacactattgccaagcgatttagatacgattctgcattggtgtctgccctcatggatatggaagaagacattctcgaagggatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagcgagcttatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaa

SEQ ID NO: 21 - DNA, 300 bp, artificial
  left HA
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgat

SEQ ID NO: 22 - DNA, 300 bp, artificial
  right HA
atattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 23 - DNA, 3428 bp, artificial
  miniAAV#5
acaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttcagctccctgtgaagtatatttccctaggaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagtaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccggtacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagtaaggagatcttcgtgcacataaataaagggggccgaccgcggcagcatttgttgagtcttacccgccgagcacaaaagcaccgacttcgagaactgaaactccaagtaaaggcgttcgcagataaggaggagggaggggacgtaaaatccgtatgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttaggaacgccgagaaagtgttgctgcctgggtatcatcactttgaatggcagcctccgctgaaaaacgtttcttcaagtaccgacgtaggtatcatagacggcctgtctggcctgtcaagcagtgtggatgattaccctgtagacactattgccaagcgatttagatacgattctgcattggtgtctgccctcatggatatggaagaagacattctcgaaggtatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagtgagcttatgcttgaactcggtggaatcctccggacgttcaagtttatttttaggggcaccggctatgacgagaaacttgttcgggaggtagagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagtaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccagcggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgcaggtttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaggacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 24 - DNA, 4028 bp, artificial; identical to SEQ ID NO: 18
  donor template AAV#5

SEQ ID NO: 25 - DNA, 3428 bp, artificial
  miniBisAAVno5
acaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagcaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccgatacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagcaaggagatcttcgtgcacataaataaagggggccgaccgcggcagcatttgttgagtcttacccgccgagcacaaaagcaccgacttcgagaactgaaactccaagttaaggcgttcgcagataaggaggagggaggggacgtaaaatccgtttgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttcgaaacgccgagaaagtgttgctgcctgggtatcatcactttgaatggcagcctccgctgaaaaacgtttcttcaagcaccgacgttggtatcatagacggcctgtctggcctgtcaagcagtgtggatgattaccctgtagacactattgccaagcgatttagatacgattctgcattggtgtctgccctcatggatatggaagaagacattctcgaagggatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagcgagcttatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 26 - DNA, 4028 bp, artificial
  alternative donor template bisAAVno5
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccagccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcgcaaacggcgggcgcaagcaagaataagcagcaaggatgtgatgaagaaaattgctaactgtagtaaaattcatctgagcaccaagttgctcgctgttgatttcccggaacatttcgtaaaaagtataagctgccaaatatgcgaacatatcctcgcagacccggttgaaaccaattgtaaacacgtattctgtcgggtttgtatactccgctgtctgaaagtgatgggctcatattgccctagctgccgatacccctgtttcccgactgatctcgaatcacctgttaagtcattccttagtgtactgaactccctcatggtgaagtgcccagccaaggaatgcaacgaagaggtttctcttgagaagtacaaccaccacattagctctcataaggagagcaaggagatcttcgtgcacataaataaagggggccgaccgcggcagcatttgttgagtcttacccgccgagcacaaaagcaccgacttcgagaactgaaactccaagttaaggcgttcgcagataaggaggagggaggggacgtaaaatccgtttgtatgacattgttcttgttggctctgcgagcgcgcaacgagcatagacaggcggatgagttggaggccatcatgcaggggaagggctcaggcctgcaaccggcagtttgccttgccatccgagttaacacttttctctcctgctctcaatatcacaagatgtacaggacagtcaaagccatcacaggccggcaaatattccagcctctccatgcacttcgaaacgccgagaaagtgttgctgcctgggtatcatcactttgaatggcagcctccgctgaaaaacgtttcttcaagcaccgacgttggtatcatagacggcctgtctggcctgtcaagcagtgtggatgattaccctgtagacactattgccaagcgatttagatacgattctgcattggtgtctgccctcatggatatggaagaagacattctcgaagggatgcgctcccaagacctggacgattatctcaatggtccgtttaccgtagtcgttaaggagagctgtgacggaatgggggatgtatcagagaagcatgggagcggacctgtagtcccagagaaagccgttcgcttttcttttacaataatgaaaataacgattgcgcattctagccagaacgttaaggtcttcgaggaagctaaacccaatagcgagctgtgttgcaaaccgctctgtcttatgctcgcggacgaatccgatcacgagactcttactgccatcctttccccactcattgcggagagagaggccatgaagtcaagcgagcttatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgctatgaggtctggcggagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 27 - DNA, 228 bp, artificial
  bGHpA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 28 - DNA, 5168 bp, artificial
  Intron 1
gtaacactcatacttttcatgccttgagccaaaatatttattacatttttatgtttctaactagaagtgcttgagctttttttccttccaggtgatgaggggatggaatgagcaaagctacatcaatttttttttaatgtatgaaaataaaaaaggtacaagaggccaagtttagggccactgaaggttcatagaaagatgcaaaatatctgaattactataaatgaatgctattgtcagaggaaaggtttaaggagtgcttcttgaatgaatgtgtacaaatcagcagaaggtaaggtgtgagactcttggaaatgaatactggtagttcaggtgagaaaaataatcaggaacataatagggtgggaggaaatgtatggtttcccaggtattaacaagtattgccaggcatttcctgaactagattggcctaagtaggagaccaatgtttctcaaaatattcactcattttagaatcactgaatgtttaaaaatgcaatttctggattccttcccaaacagccagactctttgggacctgatgatctgcatttctttttaaaaacaaactcgctcatgattctgatttgtattaattttgagaattgccatggtagagaccctgctttgaggttatgttcttgagtcaggattcctggccagggattgtgatgatatatttctctttctgaagtggttcatgcaagaggttgtctgaaggaagagcaagaattgtagtgttattttgtggatacttgagacttataaaaaggctttttattttgtcacatttttgatacatgatgtttggcaaaaaacagacgatagtatttgcagagtgaatgaataagtggaacaggtgtgataatgagaggtcacacttgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtcgagatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcacatatacagctagtctttaaaacagttttatccaaaataaatgtatcacaaaattaataaaaatagttacttgcttgttttgaataattcaaatgatacaaaaattaataaaataaaaagtgcaaaaggccctcttatcaatgccaattctatttttttcagaaattaaacactgttaagattttagtgtgtatcctttcagaattcctgtgatttcatatatgtacaaatacaaacgtatctacataaagggaatcctactatacttgctattgtcattctattctctgctttttcatgtgagcatctttccatgtcactgatgcatacagaaattgcacatatgcatcagtgcatacagaaaattaaattttctgcatggttttccactgtatgtctggaccatagtttatttaataataatgccctttgggtaattatttatattgtttcctgctttttcaaagtaacagcttttgaaacaaatctctctctgtctttatataaatattgttgcattcctgtggaaatgtttctattggataacttcccaaaaggagatttattgcatcaaagataatatattcaaaaattttaaagatattgctaaattgtctagtaggtattttataccaatttatactcctcccaagaatgtatggagatatcttaatttctccatgccttcattaatgctgaaccatataagtagttttaatctttgctaattgaatagataaaaaatatctaatctaagtctagttcttaaaagttctatcttctaccaaaagtaatacacgtctattttagggagtaaaaatcacaagtaaggataaaaaatagtgcagcaataaacacaggagtgtagatgtctctgaacatactgatttaacttcctttggataaatacccagtagtaggactgctggatcatataataattctatctttagtttttttgaggacctccatactattcttcatagtggctgtactaatttacattcctaccaactgtgtatgaaggttcccttttctctacatccttgccagcattcattattgcttgtcatttggatacaatctattttaactggggtgagatgacatctcattgtagttttgatatgcatttctctgatgatcagtggtgttgagcaccttttcatatacctgtttgccatttgtatgtcttcctttgagaaatgtctattcagatattttacctattttaaaatcggattattagattgtttcctgtagagttgtttgagctccttgtatattctggttattaatctcttgtcagatgcatagcttacaaatattttctcccatcatgtggattgtgtcttcactttgtggattgtttactttgctgtgcagaagcttttaacttgatgcaatcccatttgtccacttttgctttggttgccttccacaggagtatttaaataaatgtagtttggtagattttggtatagtaatgcaggccagtgggagtcaggggagaaatgtgtagggaagtgagatagttctaaggatcctacaaacatgccttatgattgacttactcaatgtgaaagtcaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcag

SEQ ID NO: 29 - DNA, 14 bp, artificial
  5′UTR
gtacctcagccagc

SEQ ID NO: 30 - DNA, 2781 bp, artificial
  TALEN-2-Left
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 31 - DNA, 2781 bp, artificial
  TALEN-2-Right
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 32 - DNA, 2781 bp, artificial
  TALEN-6-Left
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 33 - DNA, 2781 bp, artificial
  TALEN-6-Right
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 34 - DNA, 2781 bp, artificial
  TALEN-A-Left
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 35 - DNA, 2781 bp, artificial
  TALEN-A-Right
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 36 - DNA, 2781 bp, artificial
  TALEN-B-Left
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 37 - DNA, 2781 bp, artificial
  TALEN-B-Right
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 38 - DNA, 49 bp, artificial
  TALEN-2 target
tctatgatcagcacctaacatgatatattaaggtagaagatttaataca

SEQ ID NO: 39 - DNA, 17 bp, artificial
  TALEN-2 binding Left
tctatgatcagcaccta

SEQ ID NO: 40 - DNA, 17 bp, artificial
  TALEN-2 binding Right
tgtattaaatcttctac

SEQ ID NO: 41 - DNA, 49 bp, artificial
  TALEN-6 target
tatgatcagcacctaacatgatatattaaggtagaagatttaatacata

SEQ ID NO: 42 - DNA, 17 bp, artificial
  TALEN-6 binding Left
tatgatcagcacctaac

SEQ ID NO: 43 - DNA, 17 bp, artificial
  TALEN-6 binding Right
tatgtattaaatcttct

SEQ ID NO: 44 - DNA, 17 bp, artificial
  TALEN-A binding Left
ttttgtggaaggaaata

SEQ ID NO: 45 - DNA, 17 bp, artificial
  TALEN-A binding Right
tataagtgcttaaatat

SEQ ID NO: 46 - DNA, 17 bp, artificial
  TALEN-B binding Left
tgatatattaaggtaga

SEQ ID NO: 47 - DNA, 17 bp, artificial
  TALEN-B binding Right
ttcatgtattccaaaag

SEQ ID NO: 48 - DNA, 291 bp, artificial
  GSE56 coding sequence
atgggctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtaa

SEQ ID NO: 49 - DNA, 225 bp, artificial
  53BP1 inhibitor coding sequence
atgttgattttcgtgaaaacccttaccgggaaaaccatcaccctcgaggttgaaccctcggatacgatagaaaatgtaaaggccaagatccaggataaggaaggaattcctcctgatcagcagagactggcctttgctggcaaatcgctggaagatggacgtactttgtctgactacaatattctaaaggactctaaacttcatcctctgttgagacttcgttga

SEQ ID NO: 50 - DNA, 23 bp, artificial
  oligonucleotide TALEN-2 F
gcaaagatgaatcaaagattctg

SEQ ID NO: 51 - DNA, 21 bp, artificial
  oligonucleotide TALEN-2 R
cttcaggtgtcttttcaaagg

SEQ ID NO: 52 - DNA, 23 bp, artificial
  oligonucleotide TALEN-6 F
agcacctaatgtatactgggacc

SEQ ID NO: 53 - DNA, 20 bp, artificial
  oligonucleotide TALEN-6 R
gctggctgaggtacctgaga

SEQ ID NO: 54 - DNA, 23 bp, artificial
  oligonucleotide TALEN-A F
agcacctaatgtatactgggacc

SEQ ID NO: 55 - DNA, 20 bp, artificial
  oligonucleotide TALEN-A R
gctggctgaggtacctgaga

SEQ ID NO: 56 - DNA, 23 bp, artificial
  oligonucleotide TALEN-B F
agcacctaatgtatactgggacc

SEQ ID NO: 57 - DNA, 20 bp, artificial
  oligonucleotide TALEN-B R
gctggctgaggtacctgaga

SEQ ID NO: 58 - DNA, 23 bp, artificial
  oligonucleotide #5278
tgcagacatctcaacactttggc

SEQ ID NO: 59 - DNA, 21 bp, artificial
  oligonucleotide #4937
aaccaaccccctggaagactg

SEQ ID NO: 60 - DNA, 21 bp, artificial
  oligonucleotide #4938
ggatgaaagacccagcgtagg

SEQ ID NO: 61 - DNA, 22 bp, artificial
  oligonucleotide #4939
tgggaagacaatagcaggcatg

SEQ ID NO: 62 - DNA, 24 bp, artificial
  oligonucleotide #4940
gagattgctccagagagggtttcc

SEQ ID NO: 63 - DNA, 23 bp, artificial
  oligonucleotide #4948
aacttcagtaaagcggggacttg

SEQ ID NO: 64 - DNA, 25 bp, artificial
  oligonucleotide #5020
tgagttgtaatgcacagatcgctag

SEQ ID NO: 65 - DNA, 92 bp, Homo sapiens
ttctgtccttaaagaccttaaggtttttgtggaaggaaataaaactttacatgtatatatttaagcacttatatgtgtgtaacaagtataag

SEQ ID NO: 66 - DNA, 92 bp, Homo sapiens
ctctatgatcagcacctaacatgatatattaaggtagaagatttaatacatatcttttggaatacatgaataaataattgaatgtatttatt

SEQ ID NO: 67 - DNA, 92 bp, Homo sapiens
ttgatattggtcttaatatgacttgttttcattgttctcaggtacctcagccagcatggcagcctctttcccacccaccttgggactcagtt

SEQ ID NO: 68 - DNA, 3146 bp, artificial
  coRAG1ter
gtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccaaccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcacaaacggcgggcgcaagcaagaataagcagcaaggatgtgatgaagaaaattgctaactgtagtaaaattcatctgagcaccaagttgctcgctgttgatttccccgagcactttgtgaagagcatctcctgccagatctgtgaacacatcctggccgaccctgtggagaccaactgcaagcacgtgttttgccgggtctgcattctgcgctgtctgaaagtgatgggcagctactgcccctcttgccgatatccatgcttccctactgacctggagtctccagtgaagtcctttctgtctgtgctgaacagcctgatggtgaagtgcccagccaaggagtgtaacgaggaggtgtccctggagaagtacaatcaccacatcagctcccacaaggagtctaaggagatctttgtgcacatcaataagggcggcaggccccgccagcacctgctgagcctgaccaggagagcacagaagcacaggctgagagagctgaagctgcaagtcaaagccttcgccgacaaggaggagggaggcgatgtgaagagcgtgtgcatgacactgtttctgctggccctgagggcaaggaacgagcacaggcaggcagacgagctggaggccatcatgcagggcaagggaagcggactgcagccagccgtgtgcctggccatccgcgtgaataccttcctcagctgctctcagtaccacaagatgtatcggaccgtgaaggccatcacaggcagacagatcttccagccactgcacgcactgaggaacgcagagaaagtgctgctgccaggctaccaccactttgagtggcagccccctctgaagaatgtgtcttccagcactgatgttggcattatcgatggcctgagcggcctgtctagctccgtggacgattaccctgtggacacaatcgccaagcggttcagatatgattcagctttggtgtctgctctgatggacatggaggaggatatcctggagggcatgcggagccaggatctggacgattatctgaatggccccttcaccgtggtggtgaaggagtcctgcgacggcatgggcgacgtgagcgagaagcacggctctggccctgtggtgccagaaaaggcagtccgtttttcttttaccatcatgaagatcacaatcgcccactctagccagaacgtgaaggtgtttgaggaggccaagcccaattccgaactgtgttgcaagccactgtgcctgatgctggccgacgagagcgatcacgagaccctgacagccatcctgtcccctctcattgctgagagggaggccatgaagtcctctgagctgatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgttatgaggtctggcgtagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaa

SEQ ID NO: 69 - DNA, 3428 bp, artificial
  miniTerAAVno5
acaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccaaccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcacaaacggcgggcgcaagcaagaataagcagcaaggatgtgatgaagaaaattgctaactgtagtaaaattcatctgagcaccaagttgctcgctgttgatttccccgagcactttgtgaagagcatctcctgccagatctgtgaacacatcctggccgaccctgtggagaccaactgcaagcacgtgttttgccgggtctgcattctgcgctgtctgaaagtgatgggcagctactgcccctcttgccgatatccatgcttccctactgacctggagtctccagtgaagtcctttctgtctgtgctgaacagcctgatggtgaagtgcccagccaaggagtgtaacgaggaggtgtccctggagaagtacaatcaccacatcagctcccacaaggagtctaaggagatctttgtgcacatcaataagggcggcaggccccgccagcacctgctgagcctgaccaggagagcacagaagcacaggctgagagagctgaagctgcaagtcaaagccttcgccgacaaggaggagggaggcgatgtgaagagcgtgtgcatgacactgtttctgctggccctgagggcaaggaacgagcacaggcaggcagacgagctggaggccatcatgcagggcaagggaagcggactgcagccagccgtgtgcctggccatccgcgtgaataccttcctcagctgctctcagtaccacaagatgtatcggaccgtgaaggccatcacaggcagacagatcttccagccactgcacgcactgaggaacgcagagaaagtgctgctgccaggctaccaccactttgagtggcagccccctctgaagaatgtgtcttccagcactgatgttggcattatcgatggcctgagcggcctgtctagctccgtggacgattaccctgtggacacaatcgccaagcggttcagatatgattcagctttggtgtctgctctgatggacatggaggaggatatcctggagggcatgcggagccaggatctggacgattatctgaatggccccttcaccgtggtggtgaaggagtcctgcgacggcatgggcgacgtgagcgagaagcacggctctggccctgtggtgccagaaaaggcagtccgtttttcttttaccatcatgaagatcacaatcgcccactctagccagaacgtgaaggtgtttgaggaggccaagcccaattccgaactgtgttgcaagccactgtgcctgatgctggccgacgagagcgatcacgagaccctgacagccatcctgtcccctctcattgctgagagggaggccatgaagtcctctgagctgatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgttatgaggtctggcgtagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 70 - DNA, 4028 bp, artificial
  alternative donor template terAAVno5
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacaagctaggcccttttgctaatcatgttcatactgtcccttttttttccacaggtacctcagccagcatggcggcttcctttccccctacgctgggtctttcatccgcaccggatgaaattcaacatccacatattaagtttagtgaatggaaatttaaattgtttcgagtacgcagtttcgaaaaaactcccgaagaggctcaaaaggagaagaaggattcattcgaagggaaacctagtttggaacagagccctgctgtgctcgacaaggcggacgggcaaaaacccgtacctacccaaccgcttttgaaagcccaccccaaattcagcaagaagttccacgacaacgagaaagcgagaggcaaggcgatacaccaagcaaatctgaggcatctctgcagaatatgtggcaatagcttcagggctgacgaacacaatcgacgctacccagttcacggacccgtggatggcaaaacactggggctgcttagaaagaaagaaaagagagcgacgagttggccggatcttatcgcgaaagtcttccgaattgacgttaaggcggacgtagactctattcacccgacagagttctgtcacaattgttggtctattatgcatcgaaaattctcttctgctccctgtgaagtatatttccctcgcaatgtcacaatggagtggcatccacatactcctagctgtgacatttgtaacaccgcacgacggggccttaagaggaagtctctgcagcctaacctgcaactgtcaaaaaagttgaagacagtgctcgaccaagcccgccaagcccggcagcacaaacggcgggcgcaagcaagaataagcagcaaggatgtgatgaagaaaattgctaactgtagtaaaattcatctgagcaccaagttgctcgctgttgatttccccgagcactttgtgaagagcatctcctgccagatctgtgaacacatcctggccgaccctgtggagaccaactgcaagcacgtgttttgccgggtctgcattctgcgctgtctgaaagtgatgggcagctactgcccctcttgccgatatccatgcttccctactgacctggagtctccagtgaagtcctttctgtctgtgctgaacagcctgatggtgaagtgcccagccaaggagtgtaacgaggaggtgtccctggagaagtacaatcaccacatcagctcccacaaggagtctaaggagatctttgtgcacatcaataagggcggcaggccccgccagcacctgctgagcctgaccaggagagcacagaagcacaggctgagagagctgaagctgcaagtcaaagccttcgccgacaaggaggagggaggcgatgtgaagagcgtgtgcatgacactgtttctgctggccctgagggcaaggaacgagcacaggcaggcagacgagctggaggccatcatgcagggcaagggaagcggactgcagccagccgtgtgcctggccatccgcgtgaataccttcctcagctgctctcagtaccacaagatgtatcggaccgtgaaggccatcacaggcagacagatcttccagccactgcacgcactgaggaacgcagagaaagtgctgctgccaggctaccaccactttgagtggcagccccctctgaagaatgtgtcttccagcactgatgttggcattatcgatggcctgagcggcctgtctagctccgtggacgattaccctgtggacacaatcgccaagcggttcagatatgattcagctttggtgtctgctctgatggacatggaggaggatatcctggagggcatgcggagccaggatctggacgattatctgaatggccccttcaccgtggtggtgaaggagtcctgcgacggcatgggcgacgtgagcgagaagcacggctctggccctgtggtgccagaaaaggcagtccgtttttcttttaccatcatgaagatcacaatcgcccactctagccagaacgtgaaggtgtttgaggaggccaagcccaattccgaactgtgttgcaagccactgtgcctgatgctggccgacgagagcgatcacgagaccctgacagccatcctgtcccctctcattgctgagagggaggccatgaagtcctctgagctgatgcttgaactcggtggaatcctccggacgttcaagtttatttttcggggcaccggctatgacgagaaacttgttcgggaggttgagggattggaggcctccggtagcgtgtatatctgtaccctctgtgatgcgacccggctagaggcctctcaaaatctggtttttcactcaattacacgatctcatgctgagaacctggagcgttatgaggtctggcgtagtaatccttaccatgaaagcgtagaggagcttagagatcgagttaagggtgtcagcgccaaaccatttattgaaacggtaccctcaatagacgcccttcactgcgacatcgggaatgcggcagagttctataagatctttcagttggaaataggggaggtttataagaacccgaatgccagcaaggaggaaaggaaaaggtggcaagcgacactcgataagcatctccggaaaaagatgaacttgaagcccatcatgcgaatgaacggcaatttcgcgcggaagctgatgactaaggaaacggttgatgcggtctgcgaactgataccatcagaggaaaggcacgaggcacttagggaacttatggatctctacctgaagatgaagccagtctggcgctcatcatgcccggctaaagaatgtccggagtcactctgccaatattcttttaactcccaacggtttgcagaactgttgagcaccaaatttaagtaccgctatgaagggaaaatcacaaattatttccacaaaacactggcgcatgttccagagatcatcgaaagagacggctctataggggcgtgggcaagtgaaggaaatgaatcaggcaataaactgttccgccgatttcgcaaaatgaatgcacggcagtcaaagtgctacgaaatggaagacgttttgaagcaccattggctgtatacatccaaatatctgcaaaagtttatgaatgctcacaatgcacttaagacctctggctttaccatgaacccgcaggccagcctcggcgatccactcgggattgaagattctcttgagtcccaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag

SEQ ID NO: 71 - DNA, 3146 bp, artificial
  coRAG1quater
gtacctcagccagcatggcggcttcctttccccctacgctgggtctttcatccgcccccgatgagatccagcacccccacatcaagttttccgagtggaagtttaagctgttcagagtgcgctctttcgagaagacccctgaggaggcccagaaggagaagaaggacagctttgagggcaagccttccctggagcagtctccagccgtgctggacaaggccgatggccagaagccagtgccaacacagccactgctgaaggcacaccctaagttttctaagaagttccacgataacgagaaggcccggggcaaggcaatccaccaggccaatctgaggcacctgtgccgcatctgtggcaacagcttcagagccgatgagcacaatcggagataccctgtgcacggaccagtggacggcaagaccctgggactgctgcggaagaaggagaagagagccacaagctggcctgatctgatcgccaaagtgtttaggatcgacgtgaaggccgacgtggattctatccacccaaccgagttctgccacaactgttggagcatcatgcaccggaagtttagctccgccccatgcgaagtctatttcccccgcaacgtcaccatggagtggcaccctcacacaccatcctgcgacatctgtaacaccgcaaggaggggcctgaagaggaagtctctgcagccaaatctgcagctgagcaagaagctgaagacagtgctggatcaggcaaggcaggcaaggcagcacaagaggagagcacaggccagaatctctagcaaggacgtgatgaagaagatcgccaactgtagcaagatccacctgtccaccaagctgctggccgtggattttcccgagcacttcgtgaagagcatctcctgccaaatctgcgagcacatcctggccgaccccgtggagacaaattgcaagcacgtgttttgccgagtctgcatcctgcgctgcctgaaagtgatgggcagctactgcccctcctgtcgatatccctgctttcctaccgatctggagagccctgtgaagagcttcctgtccgtgctgaactccctgatggtgaaatgtccagccaaggagtgtaacgaggaagtgtccctggagaagtacaatcaccacatctcctctcataaagaatcaaaagagatcttcgtgcacatcaataagggcggcaggccacgccagcacctgctgtccctgacccggagggcacagaagcacaggctgagagagctgaagctgcaagtgaaggcctttgccgacaaggaggagggcggcgatgtgaagagcgtgtgcatgacactgttcctgctggccctgagggcaaggaacgagcacaggcaggcagacgagctggaggccatcatgcagggcaagggatccggactgcagccagccgtgtgcctggccatccgcgtgaatacctttctgtcttgctcccaataccacaagatgtataggaccgtgaaggccatcacaggccgccagatctttcaacctctgcacgcactgaggaacgcagagaaagtgctgctgccaggctaccaccacttcgagtggcagccccctctgaaaaatgtgtcttcctctaccgacgtcggcatcatcgatggcctgagcggcctgagctcctctgtggacgattaccccgtggatacaatcgccaagcggtttagatatgactcagctttggtgtctgccctgatggacatggaggaggatatcctggagggcatgagatcccaggacctggacgattacctgaacggccctttcaccgtggtggtgaaggagtcttgtgacggcatgggcgacgtcagcgagaagcacggaagcggaccagtggtgccagagaaggccgtgcgcttctcctttaccatcatgaaaatcacaatcgcccactcctcccaaaacgtgaaagtgttcgaggaggccaagccaaattccgagctgtgctgcaagcccctgtgcctgatgctggccgacgagagcgatcacgagaccctgacagccatcctgtccccactgatcgccgagagggaggccatgaagtctagcgagctgatgctggagctgggcggcatcctgaggaccttcaagtttatcttccgcggcaccggctacgatgagaagctcgtcagagaggtggagggactggaggcatccggaagcgtgtacatctgcaccctttgcgacgccacaaggctggaggcctcccaaaacctcgtctttcactccatcacccgctctcacgccgagaacctggagcgatacgaagtgtggagaagcaatccctatcacgagtccgtggaggagctgagggatcgcgtgaagggcgtgtctgccaagcctttcatcgagacagtgccaagcatcgacgccctgcactgtgatatcggcaacgccgccgagttttacaagatcttccaactggagatcggcgaagtctataagaacccaaatgccagcaaggaggagcggaagagatggcaggccaccctggacaagcacctgcggaagaagatgaatctgaagcccatcatgcggatgaacggcaatttcgccagaaagctgatgaccaaggagacagtggatgccgtgtgcgagctgatcccatccgaggagaggcacgaggccctgagggagctgatggacctgtacctgaagatgaagcccgtgtggcggtcctcttgccctgccaaagagtgtcctgagagcctgtgccagtattctttcaattcacaacgttttgctgagctgctgtccaccaaatttaaataccgctatgagggcaagatcaccaattacttccacaagacactggcccacgtgcctgagatcatcgagagagatggaagcatcggagcatgggcatccgagggaaacgagtctggcaataagctgtttcggagattccggaagatgaacgccagacagtctaagtgctatgagatggaggacgtgctgaagcaccactggctgtacacaagcaaatatctgcagaagtttatgaacgcccacaatgccctgaagacctccggcttcacaatgaatccacaggcatctctgggcgaccctctgggaatcgaggattccctggagtctcaagacagcatggagttctaa

SEQ ID NO: 72 - DNA, 3428 bp, artificial
  miniQuaterAAVno5
acaagctaggcccttttgctaatcatgttcatactgtcccttttttttccacaggtacctcagccagcatggcggcttcctttccccctacgctgggtctttcatccgcccccgatgagatccagcacccccacatcaagttttccgagtggaagtttaagctgttcagagtgcgctctttcgagaagacccctgaggaggcccagaaggagaagaaggacagctttgagggcaagccttccctggagcagtctccagccgtgctggacaaggccgatggccagaagccagtgccaacacagccactgctgaaggcacaccctaagttttctaagaagttccacgataacgagaaggcccggggcaaggcaatccaccaggccaatctgaggcacctgtgccgcatctgtggcaacagcttcagagccgatgagcacaatcggagataccctgtgcacggaccagtggacggcaagaccctgggactgctgcggaagaaggagaagagagccacaagctggcctgatctgatcgccaaagtgtttaggatcgacgtgaaggccgacgtggattctatccacccaaccgagttctgccacaactgttggagcatcatgcaccggaagtttagctccgccccatgcgaagtctatttcccccgcaacgtcaccatggagtggcaccctcacacaccatcctgcgacatctgtaacaccgcaaggaggggcctgaagaggaagtctctgcagccaaatctgcagctgagcaagaagctgaagacagtgctggatcaggcaaggcaggcaaggcagcacaagaggagagcacaggccagaatctctagcaaggacgtgatgaagaagatcgccaactgtagcaagatccacctgtccaccaagctgctggccgtggattttcccgagcacttcgtgaagagcatctcctgccaaatctgcgagcacatcctggccgaccccgtggagacaaattgcaagcacgtgttttgccgagtctgcatcctgcgctgcctgaaagtgatgggcagctactgcccctcctgtcgatatccctgctttcctaccgatctggagagccctgtgaagagcttcctgtccgtgctgaactccctgatggtgaaatgtccagccaaggagtgtaacgaggaagtgtccctggagaagtacaatcaccacatctcctctcataaagaatcaaaagagatcttcgtgcacatcaataagggcggcaggccacgccagcacctgctgtccctgacccggagggcacagaagcacaggctgagagagctgaagctgcaagtgaaggcctttgccgacaaggaggagggcggcgatgtgaagagcgtgtgcatgacactgttcctgctggccctgagggcaaggaacgagcacaggcaggcagacgagctggaggccatcatgcagggcaagggatccggactgcagccagccgtgtgcctggccatccgcgtgaatacctttctgtcttgctcccaataccacaagatgtataggaccgtgaaggccatcacaggccgccagatctttcaacctctgcacgcactgaggaacgcagagaaagtgctgctgccaggctaccaccacttcgagtggcagccccctctgaaaaatgtgtcttcctctaccgacgtcggcatcatcgatggcctgagcggcctgagctcctctgtggacgattaccccgtggatacaatcgccaagcggtttagatatgactcagctttggtgtctgccctgatggacatggaggaggatatcctggagggcatgagatcccaggacctggacgattacctgaacggccctttcaccgtggtggtgaaggagtcttgtgacggcatgggcgacgtcagcgagaagcacggaagcggaccagtggtgccagagaaggccgtgcgcttctcctttaccatcatgaaaatcacaatcgcccactcctcccaaaacgtgaaagtgttcgaggaggccaagccaaattccgagctgtgctgcaagcccctgtgcctgatgctggccgacgagagcgatcacgagaccctgacagccatcctgtccccactgatcgccgagagggaggccatgaagtctagcgagctgatgctggagctgggcggcatcctgaggaccttcaagtttatcttccgcggcaccggctacgatgagaagctcgtcagagaggtggagggactggaggcatccggaagcgtgtacatctgcaccctttgcgacgccacaaggctggaggcctcccaaaacctcgtctttcactccatcacccgctctcacgccgagaacctggagcgatacgaagtgtggagaagcaatccctatcacgagtccgtggaggagctgagggatcgcgtgaagggcgtgtctgccaagcctttcatcgagacagtgccaagcatcgacgccctgcactgtgatatcggcaacgccgccgagttttacaagatcttccaactggagatcggcgaagtctataagaacccaaatgccagcaaggaggagcggaagagatggcaggccaccctggacaagcacctgcggaagaagatgaatctgaagcccatcatgcggatgaacggcaatttcgccagaaagctgatgaccaaggagacagtggatgccgtgtgcgagctgatcccatccgaggagaggcacgaggccctgagggagctgatggacctgtacctgaagatgaagcccgtgtggcggtcctcttgccctgccaaagagtgtcctgagagcctgtgccagtattctttcaattcacaacgttttgctgagctgctgtccaccaaatttaaataccgctatgagggcaagatcaccaattacttccacaagacactggcccacgtgcctgagatcatcgagagagatggaagcatcggagcatgggcatccgagggaaacgagtctggcaataagctgtttcggagattccggaagatgaacgccagacagtctaagtgctatgagatggaggacgtgctgaagcaccactggctgtacacaagcaaatatctgcagaagtttatgaacgcccacaatgccctgaagacctccggcttcacaatgaatccacaggcatctctgggcgaccctctgggaatcgaggattccctggagtctcaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 73 - DNA, 4028 bp, artificial
  alternative donor template terAAVno5
tgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatacaagctaggcccttttgctaatcatgttcatactgtcccttttttttccacaggtacctcagccagcatggcggcttcctttccccctacgctgggtctttcatccgcccccgatgagatccagcacccccacatcaagttttccgagtggaagtttaagctgttcagagtgcgctctttcgagaagacccctgaggaggcccagaaggagaagaaggacagctttgagggcaagccttccctggagcagtctccagccgtgctggacaaggccgatggccagaagccagtgccaacacagccactgctgaaggcacaccctaagttttctaagaagttccacgataacgagaaggcccggggcaaggcaatccaccaggccaatctgaggcacctgtgccgcatctgtggcaacagcttcagagccgatgagcacaatcggagataccctgtgcacggaccagtggacggcaagaccctgggactgctgcggaagaaggagaagagagccacaagctggcctgatctgatcgccaaagtgtttaggatcgacgtgaaggccgacgtggattctatccacccaaccgagttctgccacaactgttggagcatcatgcaccggaagtttagctccgccccatgcgaagtctatttcccccgcaacgtcaccatggagtggcaccctcacacaccatcctgcgacatctgtaacaccgcaaggaggggcctgaagaggaagtctctgcagccaaatctgcagctgagcaagaagctgaagacagtgctggatcaggcaaggcaggcaaggcagcacaagaggagagcacaggccagaatctctagcaaggacgtgatgaagaagatcgccaactgtagcaagatccacctgtccaccaagctgctggccgtggattttcccgagcacttcgtgaagagcatctcctgccaaatctgcgagcacatcctggccgaccccgtggagacaaattgcaagcacgtgttttgccgagtctgcatcctgcgctgcctgaaagtgatgggcagctactgcccctcctgtcgatatccctgctttcctaccgatctggagagccctgtgaagagcttcctgtccgtgctgaactccctgatggtgaaatgtccagccaaggagtgtaacgaggaagtgtccctggagaagtacaatcaccacatctcctctcataaagaatcaaaagagatcttcgtgcacatcaataagggcggcaggccacgccagcacctgctgtccctgacccggagggcacagaagcacaggctgagagagctgaagctgcaagtgaaggcctttgccgacaaggaggagggcggcgatgtgaagagcgtgtgcatgacactgttcctgctggccctgagggcaaggaacgagcacaggcaggcagacgagctggaggccatcatgcagggcaagggatccggactgcagccagccgtgtgcctggccatccgcgtgaatacctttctgtcttgctcccaataccacaagatgtataggaccgtgaaggccatcacaggccgccagatctttcaacctctgcacgcactgaggaacgcagagaaagtgctgctgccaggctaccaccacttcgagtggcagccccctctgaaaaatgtgtcttcctctaccgacgtcggcatcatcgatggcctgagcggcctgagctcctctgtggacgattaccccgtggatacaatcgccaagcggtttagatatgactcagctttggtgtctgccctgatggacatggaggaggatatcctggagggcatgagatcccaggacctggacgattacctgaacggccctttcaccgtggtggtgaaggagtcttgtgacggcatgggcgacgtcagcgagaagcacggaagcggaccagtggtgccagagaaggccgtgcgcttctcctttaccatcatgaaaatcacaatcgcccactcctcccaaaacgtgaaagtgttcgaggaggccaagccaaattccgagctgtgctgcaagcccctgtgcctgatgctggccgacgagagcgatcacgagaccctgacagccatcctgtccccactgatcgccgagagggaggccatgaagtctagcgagctgatgctggagctgggcggcatcctgaggaccttcaagtttatcttccgcggcaccggctacgatgagaagctcgtcagagaggtggagggactggaggcatccggaagcgtgtacatctgcaccctttgcgacgccacaaggctggaggcctcccaaaacctcgtctttcactccatcacccgctctcacgccgagaacctggagcgatacgaagtgtggagaagcaatccctatcacgagtccgtggaggagctgagggatcgcgtgaagggcgtgtctgccaagcctttcatcgagacagtgccaagcatcgacgccctgcactgtgatatcggcaacgccgccgagttttacaagatcttccaactggagatcggcgaagtctataagaacccaaatgccagcaaggaggagcggaagagatggcaggccaccctggacaagcacctgcggaagaagatgaatctgaagcccatcatgcggatgaacggcaatttcgccagaaagctgatgaccaaggagacagtggatgccgtgtgcgagctgatcccatccgaggagaggcacgaggccctgagggagctgatggacctgtacctgaagatgaagcccgtgtggcggtcctcttgccctgccaaagagtgtcctgagagcctgtgccagtattctttcaattcacaacgttttgctgagctgctgtccaccaaatttaaataccgctatgagggcaagatcaccaattacttccacaagacactggcccacgtgcctgagatcatcgagagagatggaagcatcggagcatgggcatccgagggaaacgagtctggcaataagctgtttcggagattccggaagatgaacgccagacagtctaagtgctatgagatggaggacgtgctgaagcaccactggctgtacacaagcaaatatctgcagaagtttatgaacgcccacaatgccctgaagacctccggcttcacaatgaatccacaggcatctctgggcgaccctctgggaatcgaggattccctggagtctcaagacagcatggagttctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaag